(19) United States
(12) Patent Application Publication
(10) Pub. No.: US 2024/0099947 A1
(43) Pub. Date: Mar. 28, 2024

(54) Abrasive Silica Particles

(71) Applicant: PQ SILICAS UK LIMITED, Warrington Cheshire (GB)

(72) Inventors: Simon Stebbing, Warrington Cheshire (GB); Pam Gleaves, Warrington Cheshire (GB); Ian McKeown, Liverpool Merseyside (GB); Anthony Nock, Crown Point, IN (US); Ufuk Senturk, Gladwyne, PA (US)

(21) Appl. No.: 18/256,546

(22) PCT Filed: Dec. 10, 2021
(86) PCT No.: PCT/GB2021/053239

§ 371 Date: Jun. 8, 2023

Related U.S. Application Data
(60) Provisional application No. 63/124,431, filed on Dec. 11, 2020.

(30) Foreign Application Priority Data
Jan. 15, 2021 (GB) 2100521.0

(51) Int. Cl. A61K 8/25; A61K 8/02; A61Q 11/00

(52) U.S. Cl. CPC A61K 8/25; A61K 8/0275; A61Q 11/00; A61K 2800/28; A61K 2800/651

(57) ABSTRACT

Described are abrasive silica particles suitable for use in a dentifrice composition, the particles comprising particles of a first abrasive silica in an amount of at least 90 wt % relative to the total weight of the abrasive silica particles; and particles of a second abrasive silica in an amount of up to 10 wt % relative to the total weight of the abrasive silica particles, wherein the weight median particle diameter (d50) of the particles of the second abrasive silica is less than the weight median particle diameter (d50) of the particles of the first abrasive silica and from 15% to 70% of the d50 value of the particles of the first abrasive silica. Dentifrice compositions comprising such particles and methods of preparing such particles are also described.

### FIELD OF THE INVENTION

The present invention relates to abrasive silica particles for use in dentifrice applications and particularly to silica compositions comprising particles of a first abrasive silica, and particles of a second abrasive silica. The first abrasive silica is different to the second abrasive silica. Abrasive silica particles described herein provide desirable cleaning and abrasion properties when incorporated into dentifrice compositions such as toothpaste.

### BACKGROUND

Dentifrices are used in oral hygiene, and particularly for cleaning teeth. Abrasive silicas have been provided in dentifrice formulations, particularly in toothpastes, as a primary cleaning agent to provide cleaning to the surface of teeth. Cleaning may include complete or partial removal of food particles, plaque, stains, bacteria/biofilms and the like from the surface of teeth. Thickeners, which may be non-abrasive silicas, may also be provided in dentifrice formulations.

The abrasiveness of a silica and its ability to provide cleaning properties are related. As a broad generalisation, a more abrasive silica will tend to provide a higher degree of cleaning than a less abrasive silica. However, highly abrasive silicas are also more likely to damage the tooth surface than less abrasive silicas. The skilled dentifrice practitioner must therefore strike a balance between abrasiveness and cleaning, such that dentifrice products, including toothpastes, gels, or powders, provide effective cleaning of the surface of a user's teeth whilst causing minimal abrasive damage to the teeth.

Dentifrice compositions (e.g. toothpaste) containing a first “bulk” abrasive silica, together with a relatively smaller amount of a second abrasive silica are known. Typically, two silicas are used to modify the properties, e.g. cleaning properties, of a dentifrice composition containing the combination of silicas. It is conventional for commercial dentifrice to provide abrasive silica particles comprising around 20 wt % or more of a second abrasive silica and usually up to around 80 wt % of the first “bulk” abrasive silica to achieve acceptable cleaning properties in combination with acceptable abrasion properties. Usually, such second silicas may comprise fine/highly milled silica particles. Such second silicas are also typically more abrasive than the first “bulk” silica. In turn, the abrasive silica particles (containing a first “bulk” silica and a lesser amount of second silica) are incorporated into dentifrices in an amount conventionally around 10 to 20 wt % relative to the total weight of the dentifrice.

Abrasive silica compositions comprising silica gel abrasives and/or precipitated silicas for use in dentifrices are described in U.S. Pat. No. 6,896,876, US 2001/0055572, U.S. Pat. Nos. 5,651,958, and 5,658,553. WO2005/065634 describes an abrasive system comprising crystalline aluminosilicate, and at least one abrasive amorphous silica, optionally two. These documents describe abrasive silica particles/abrasive systems comprising abrasive silica particles, wherein the second silica is preferably provided in the abrasive silica particles in an amount exceeding 10 wt % of the overall abrasive silica composition.

### SUMMARY OF INVENTION

The present invention has particular application to dentifrice compositions that comprise a first “bulk” abrasive silica and a second abrasive silica, where the second abrasive silica is used to enhance the performance of the dentifrice relative to dentifrices containing the first silica alone.

Abrasive silica particles and abrasive silica compositions are described, which provide desirable cleaning performance that is acceptable for use in dentifrices. The inventors have discovered that, by carefully controlling certain characteristic properties of the respective first “bulk” abrasive silica relative to the second abrasive silica, abrasive silica particles or abrasive silica compositions can be provided which exhibit desirable cleaning performance and abrasive properties relative to comparative mixed-silica dentifrice compositions, whilst using considerably lower amounts of the second abrasive silica than amounts conventionally proposed in the art. As described in the examples, the silicas and dentifrice compositions of the invention provide surprisingly effective cleaning and abrasive properties despite containing the second abrasive silica in only low quantities relative to the first “bulk” abrasive silica, often in quantities far below 10% wt, e.g. as low as 1% wt in some cases. In other words, by carefully controlling certain parameters of the respective first “bulk” abrasive and second abrasive silicas, the inventors have discovered that the second abrasive silica can be provided in considerably lower amounts than previously thought to be effective, but without the expected detrimental effect to the cleaning properties of the silica.

This unexpected benefit has clear technical and commercial advantages for industrial dentifrice applications, not least because second abrasive silicas in such commercial applications are typically desired to be more abrasive and have a smaller average particle size compared to the first “bulk” abrasive silicas. This in turn tends to render them more difficult, energy intensive and time consuming to manufacture relative to typical bulk dentifrice silicas. As such, second abrasive silicas are usually the more valuable silica component in the combination due to the more extensive milling and processing required to generate such silicas from regular silica feedstocks. Thus, embodiments described herein are able to deliver desirable cleaning/abrasion performance whilst advantageously requiring less of the valuable/energy intensive silica feedstocks. Ultimately, this means the methods of manufacturing the compositions herein are less energy intensive compared to conventional multi-silica particles for dentifrice applications. Moreover, the lower amount of second abrasive silica required can have beneficial effects on reducing abrasion without meaningfully affecting cleaning performance.

Particularly, by carefully controlling the relative particle size ratio of the second abrasive silica and first “bulk” abrasive silica (and optionally further controlling the abrasiveness of the second abrasive silica relative to the first “bulk” abrasive silica) and/or the oil absorption value of the second abrasive silica and first “bulk” abrasive silica, it is possible to provide the beneficial cleaning performance mentioned above.

In a first aspect of the present invention, there is provided abrasive silica particles suitable for use in a dentifrice composition. The particles comprise particles of a first abrasive silica in an amount of at least 90 wt % relative to the total weight of the abrasive silica particles; and particles of a second abrasive silica in an amount of up to 10 wt % relative to the total weight of the abrasive silica particles. The weight median particle diameter (d₅₀) of the particles of the second abrasive silica is less than the weight median particle diameter (d₅₀) of the particles of the first abrasive silica, and is from 15% to 70% of the d₅₀ value of the particles of the first abrasive silica.

As is evident in the data disclosed herein, it has been surprisingly found that, by controlling the respective parameters of the second abrasive silica relative to the first silica, relatively low amounts of the second abrasive silica relative to the first silica can be used without detriment to the cleaning performance.

In a second aspect of the present invention, there is provided a composition for use in a dentifrice, the composition comprising the abrasive silica particles according to the first aspect of the invention, and optionally a carrier.

In a third aspect of the present invention, there is provided a dentifrice comprising abrasive silica particles according to the first aspect of the present invention; or a composition according to the second aspect of the invention.

In a fourth aspect of the present invention, there is provided a method of preparing abrasive silica particles suitable for use in a dentifrice composition, the method comprising combining particles of a first abrasive silica and particles of a second abrasive silica to provide the abrasive silica particles. The particles of the first abrasive silica are present in an amount of at least 90 wt % relative to the total weight of the abrasive silica particles. The particles of the second abrasive silica are present in an amount of up to wt % relative to the total weight of the abrasive silica particles. The weight median particle diameter (d₅₀) of the second abrasive silica is less than the weight median particle diameter (d₅₀) of the first abrasive silica, and is from 15% to 70% of the d₅₀ value of the first silica particles.

In a fifth aspect of the present invention, there is provided abrasive silica particles prepared according to the method according to the fourth aspect of the present invention. The abrasive silica particles may be as described according to the first aspect of the invention.

### LIST OF FIGURES

The present invention will now be described by way of example only with reference to the accompanying Figures, in which:

FIG. 1 is a graph illustrating the cleaning performance of Examples 1-31 described herein as determined by the Ferric Tannate cleaning test at 100 brush strokes (FT₁₀₀).

FIG. 2 is a graph illustrating the FT₁₀₀ cleaning performance relative to plastic abrasion value (PAV) for Examples 1-31 described herein.

### DETAILED DESCRIPTION

Abrasive silica particles in accordance with the present invention are eminently suitable for use in dentifrice compositions.

Unless stated otherwise herein, references to “abrasive silica particles” according to the invention and references to properties of such particles (e.g. abrasion characteristics described by way of PAV or RDA parameters, or oil absorption characteristics, or FT₁₀₀ cleaning values) are intended to refer to the entire population of abrasive silicas particles as described. Where particular sub-populations of abrasive particles and properties of such of sub-populations of particles are intended to be referred to, this is stated herein, e.g. where the text refers to first, second and/or further abrasive silicas.

The phrases “particles of first (or second or third or further) abrasive silica”, “first (or second or third or further) silica”, and “first (or second or third or further) silica particles” are used herein interchangeably. The skilled person will appreciate that these terms refer to particles of first or second (and optionally third or further) silicas included (i.e. as sub-populations) within the populations of abrasive silica particles according to the present invention. Particles of ‘further’ abrasive silica may be a fourth, fifth, sixth etc. abrasive silica.

It will be appreciated that the particles of first (or second or third or further) abrasive silica described herein may contain modest amounts of water. In typical embodiments, the abrasive silicas (the first/second/third abrasive silicas) may each independently comprise no more than 8 wt % water, optionally no more than 6 wt % water. Unless otherwise specified herein, wt % as recited herein refers to the total weight basis. It will be appreciated that alternatively, wt % may be calculated on the dry weight basis of the abrasive silicas (the first/second/third abrasive silicas).

In the present disclosure, references to first, second, optionally third (optionally still further e.g. fourth, etc.) abrasive silicas is intended to refer to different abrasive silicas contained within the population of abrasive silica particles of the invention. The skilled person will understand that different silicas may have different characteristics (e.g. chemical or physical characteristics) and will be readily able to propose suitable silicas for use in the invention. The silicas may for instance differ in pore properties, surface area, hardness, and/or acidity. Different silicas may for instance be prepared by different synthetic methods. A person skilled in the art would be readily able to select first, second, optionally third, and optionally still further (e.g. fourth) abrasive silicas that are eminently suitable for use in the present invention, for example from commercially available silicas. The skilled person would additionally be able to prepare first, second, optionally third, and optionally still further (e.g. fourth) abrasive silicas for use in the present invention using routine methods known to those skilled in the art.

The first and second (optionally third, optionally further e.g. fourth) silica particles may be selected from any suitable type of abrasive silica, provided such silicas have the particle properties as defined herein. In embodiments, the first, second (and optionally third, and optionally further e.g. fourth) abrasive silicas may be selected from precipitated silicas and silica gels. Typically, at least the first silica will be a precipitated silica and in embodiments, the first and second (and optionally third, and optionally further) silicas are selected from precipitated silicas. It will be appreciated that precipitated silicas are typically amorphous. The first silica may be an abrasive silica gel, and in embodiments, the second (and optionally third, and optionally further) silica is selected from abrasive silica gels, and precipitated silicas.

In effect, the present claims describe abrasive silica particles (i.e. a population of abrasive silica particles) containing a combination of two (or optionally three, or optionally more e.g. four, five, etc.) different abrasive silica sub-populations, wherein it has been observed that controlling particular relative particle properties (e.g. weight median particle size d₅₀) of the first and the second (and optionally the third, optionally still further e.g. fourth) abrasive silicas provides the benefits described herein.

In accordance with the present invention, particles of first abrasive silica are provided in at least 90 wt % relative to the total weight of the abrasive silica particles, and are thus sometimes described herein as a first “bulk” silica or first “bulk” silica particles or a first “bulk” abrasive silica or particles of a first “bulk” abrasive silica. Particles of the second abrasive silica are a minor component of the overall total weight of abrasive silica particles, being provided in an amount up to 10 wt % relative to the total weight of the abrasive silica particles, e.g. up to 7 wt %.

In embodiments, the abrasive silica particles according to the present invention may comprise second silica particles in an amount of from 0.05 wt % to 10 wt % relative to the total weight of the abrasive silica particles, e.g. from 1 wt % to 10 wt %. The abrasive silica particles may comprise second silica particles in an amount of from 2 to 7 wt % relative to the total weight of the abrasive silica particles, optionally from 3 to 5 wt % (e.g. around 4 wt %) relative to the total weight of the abrasive silica particles.

In embodiments, the abrasive silica particles according to the present invention further comprise particles of a third abrasive silica, wherein the total combined weight of the particles of the second and third abrasive silicas does not exceed 10 wt % relative to the total weight of the abrasive silica particles. Particles of the second abrasive silica and particles of the third abrasive silica are therefore a minor component of the overall total weight of abrasive silica particles, being provided in an amount up to 10 wt % relative to the total weight of the abrasive silica particles.

In embodiments, the abrasive silica particles according to the present invention may comprise second and third silica particles in a total combined amount of from 0.05 wt % to 10 wt % relative to the total weight of the abrasive silica particles, e.g. from 1 wt % to wt %. The abrasive silica particles may comprise second and third silica particles in a total combined amount of from 1 to 10 wt %, optionally 2 to 7 wt % relative to the total weight of the abrasive silica particles, optionally from 3 to 5 wt % relative to the total weight of the abrasive silica particles.

In embodiments, the first abrasive silica is an amorphous precipitated silica and/or the second abrasive silica is an amorphous precipitated silica and/or the optional third and/or optionally still further (e.g. fourth) abrasive silica is an amorphous precipitated silica. The first abrasive silica may be an amorphous precipitated silica. The second abrasive silica may be an amorphous precipitated silica. The optional third abrasive silica may be an amorphous precipitated silica. In embodiments, each (e.g. first, second and third) abrasive silica may be an amorphous precipitated silicas.

In embodiments, the abrasive silica particles according to the present invention consist essentially of the first and second silica particles, and optionally the third silica particles. ‘Consisting essentially of’, as used herein, means substantially free of other components e.g. in embodiments the abrasive silica particles may comprise the first silica particles and the second silica particles and optionally the third (and optionally further e.g. fourth) silica particles in an amount of at least 95 wt % relative to the overall weight of the abrasive silica particles, optionally at least 98 wt %, optionally still at least 99 wt %. In embodiments, the abrasive silica particles according to the present invention may consist of only the first silica particles and the second silica particles and optionally the third (and optionally further e.g. fourth) silica particles, e.g. where such particles make up 100 wt % of the abrasive silica particles.

In embodiments, the abrasive silica particles according to the present invention comprise particles of a first abrasive silica in an amount of at least 90 wt % relative to the total weight of the abrasive silica particles; and particles of a second abrasive silica in an amount of from 1 to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica.

In embodiments, the abrasive silica particles according to the present invention comprise particles of a first abrasive silica in an amount of at least 90 wt % relative to the total weight of the abrasive silica particles; and particles of a second abrasive silica in an amount of from 2 to 7 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica.

In embodiments, the abrasive silica particles according to the present invention comprise particles of a first abrasive silica in an amount of at least 90 wt % relative to the total weight of the abrasive silica particles; and particles of a second abrasive silica in an amount of from 3 to 5 wt % (e.g. around 4 wt %) relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica.

Particle Size

The size of the particles may be characterised by weight median particle diameter (d₅₀). This refers to wherein 50% by weight of particles comprised in a particle population (e.g. the abrasive silica, such as particles of the first abrasive silica) have a particle diameter equal to the d₅₀ value, or less. Further characterisation of the particle size distribution of a given population of particles can be provided by defining the proportion of particles that have a particular diameter or less. For example, d₉₀ refers to wherein 90% by weight of particles comprised in a particle population (e.g. the abrasive silica, such as particles of the first abrasive silica) have a particle diameter equal to the d₉₀ value, or less. For example, d₁₀ refers to wherein 10% by weight of particles comprised in a particle population (e.g. the abrasive silicas, such as the particles of the first abrasive silica) have a diameter equal to the d₁₀ value, or less.

In embodiments, the abrasive silica particles according to the present invention have a weight median particle diameter (d₅₀) value of 5 μm or more, preferably 9 μm or more. The abrasive silica particles may have a d₅₀ value of 15 μm or less, preferably 12 μm or less. The abrasive silica particles according to the present invention have a weight median particle diameter (d₅₀) value from 5 μm to 15 μm, optionally from 9 μm to 12 μm (e.g. around 10 μm, around 11 μm).

The abrasive silica particles may have a d₉₀ value (wherein 90% by weight of particles comprised in the abrasive silica particles has a diameter less than the d₉₀ value) of 25 μm or more. The abrasive silica particles may have a d₉₀ value of 35 μm or less. The abrasive silica particles according to the present invention have a d₅₀ value from 25 μm to 35 μm, optionally from 30 μm to 34 μm (e.g. around 31 μm, around 32 μm).

The abrasive silica particles may have a d₁₀ value (wherein 10% by weight of particles comprised in the abrasive silica particles has a diameter less than the d₁₀ value) of 2 μm or more. The abrasive silica particles may have a d₁₀ value of 4 μm or less. The abrasive silica particles according to the present invention have a d₁₀ value from 2 μm to 4 μm (e.g. around 3 μm).

In embodiments, the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein, the weight median particle diameter (d₅₀) of the second silica particles is from 15% to 65% of the d₅₀ of the first silica particles; optionally from 15% to 55% of the d₅₀ value of the first silica particles. In embodiments, the weight median particle diameter (d₅₀) of the second silica particles is from 15% to 50% of the d₅₀ of the first silica particles, optionally from 20% to 50% of the d₅₀ value of the first silica particles.

In embodiments, the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the second silica particles is from 15% to 25% of the d₅₀ of the first silica particles; optionally from 18% to 23% (e.g. around 20, around 21, around 22) of the d₅₀ value of the first silica particles.

In embodiments, the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the second silica particles is from 25% to 40% (e.g. around 35, around 36) of the d₅₀ of the first silica particles; optionally from 30% to 35% (e.g. around 31, around 32, around 33) of the d₅₀ value of the first silica particles.

In embodiments, the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the second silica particles is from 40% to 55% of the d₅₀ of the first silica particles; optionally from 40% to 50% (e.g. around 44, around 45, around 46, around 47) of the d₅₀ value of the first silica particles.

In embodiments, the d₅₀ of the particles of the first abrasive silica may be less than 15 μm, optionally less than 13 μm, and optionally still less than 12 μm. The d₅₀ of the particles of the first abrasive silica is generally 5 μm or more, optionally 7 μm or more, and optionally still 9 μm or more. The d₅₀ of the particles of the first abrasive silica may for instance be from 5 to 15 μm, optionally from 7 to 14 μm, optionally still from 8 to 13 μm, further optionally still from 9 to 12 μm. In embodiments, the d₅₀ of the particles of the particles of the first abrasive silica is from 10 to 12 μm (e.g. about 10 μm, about 11 μm, about 12 μm).

The particles of the first abrasive silica may have a d₉₀ value, wherein 90% by weight of particles comprised in the first silica particles has a diameter less than the d₉₀ value, of 35 μm or less. The d₉₀ of the particles of the first abrasive silica is generally 20 μm or more, optionally 23 μm or more, and optionally still 25 μm or more. The d₉₀ of the particles of the first abrasive silica may be from 25 μm to 35 μm, optionally from 27 to 33 μm, optionally still from 28 to 32 μm.

The particles of the first abrasive silica may have a d₁₀ value, wherein 10% by weight of particles comprised in the first silica particles has a diameter less than the d₁₀ value, of 4 μm or less. The d₁₀ of the particles of the first abrasive silica may generally be 1 μm or more, optionally 2 μm or more. The d₁₀ of the particles of the first abrasive silica may be from 1 μm to 4 μm, optionally from 1.5 to 3.5 μm, optionally still from 2 to 3.5 μm, or 2-4 μm.

In embodiments, the d₅₀ of the particles of the second abrasive silica is 9 μm or less, optionally 8.5 μm or less, optionally still 8 μm or less. The d₅₀ of the particles of the second abrasive silica may be 0.5 μm or more, optionally 1 μm or more, and optionally still 1.5 μm or more, further optionally still 2 μm or more. In embodiments, the d₅₀ of the particles of the second abrasive silica is from 1 to 9 μm, optionally from 1.5 to 8.5 μm, optionally still from 2 to 8 μm, further optionally still from 2 to 7 μm. In embodiments, the d₅₀ of the particles of the particles of the second abrasive silica is from 2 to 6 μm, optionally still from 2 to 5 μm.

The particles of the second abrasive silica may have a d₉₀ value, wherein 90% by weight of particles comprised in the particles of the second abrasive silica has a diameter less than the d₉₀ value, of 25 μm or less, optionally 20 μm or less. The d₉₀ of the particles of the second abrasive silica may be 2 μm or more, optionally 2.5 μm or more, optionally still 3 μm or more, further optionally still 3.5 μm or more. The d₉₀ of the particles of the second abrasive silica may be from 3 μm to 25 μm, optionally from 3 to 20 μm, optionally still from 3 to 15 μm, further optionally still from 3 to 13 μm.

The particles of the second abrasive silica may have a d₁₀ value, wherein 10% by weight of particles comprised in the particles of the second abrasive silica has a diameter less than the d₁₀ value, of 5 μm or less, optionally of 4 μm or less, optionally still of 3 μm, optionally still of 2.5 μm or less. The d₁₀ of the particles of the second abrasive silica may be 0.5 μm or more, optionally 1 μm or more. The d₁₀ of the particles of the second abrasive silica may be from 0.5 μm to 5 μm, optionally from 0.5 to 4 μm, optionally still from 1 to 3 μm, further optionally still from 1 to 2.5 μm.

In embodiments, the abrasive silica particles comprise first silica particles having a d₅₀ value of from 5 μm to 15 μm, and a d₉₀ value, wherein 90% by weight of particles comprised in the first silica has a diameter less than the d₉₀ value, of from 25 μm to 35 μm; and second silica particles having a d₅₀ value of from 2 μm to 6 μm, and a d₉₀ value of from 3 μm to 25 μm.

In embodiments, the abrasive silica particles of the present invention optionally further comprise particles of a third, and optionally still further (e.g. fourth, fifth, etc.) abrasive silica. The optional particles of a third abrasive silica may have a d₁₀, d₅₀ and d₉₀ in accordance with the d₁₀, d₅₀ and d₉₀ values outlined above in respect of the second abrasive silica. The optional particles of third abrasive silica may have a d₅₀ relationship with the first abrasive silica that is in accordance with the d₅₀ relationship of the second abrasive silica and the first abrasive silica described above.

In embodiments, the abrasive silica particles of the present invention further comprise particles of a third abrasive silica, wherein the weight median particle diameter (d₅₀) of the particles of the third silica is less than the d₅₀ of the first silica particles and from 15% to 70% of the d₅₀ of the first silica particles, wherein the combined weight of the particles of the second and third abrasive silicas does not exceed 10 wt % relative to the total weight of the abrasive silica particles.

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica; the first silica particles having a d₅₀ value of from 5 μm to 15 μm; and second silica particles having a d₅₀ value of from 1 μm to 9 μm.

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 25% (e.g. around 20%, around 21%) of the d₅₀ value of the particles of the first abrasive silica; the first silica particles having a d₅₀ value of from 10 μm to 12 μm (preferably around 11 μm); and the second silica particles having a d₅₀ value of from 1 μm to 3 μm (preferably around 2 μm).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 25% to 40% (e.g. around 33%, around 34%, around 35%, around 36%) of the d₅₀ value of the particles of the first abrasive silica; the first silica particles having a d₅₀ value of from 10 μm to 12 μm (preferably around 11 μm); and the second silica particles having a d₅₀ value of from 3 μm to 5 μm (preferably around 4 μm).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 40% to 55% (e.g. around 44, around 45, around 46, around 47, around 48, around 49, around 50) of the d₅₀ value of the particles of the first abrasive silica; the first silica particles having a d₅₀ value of from 10 μm to 12 μm (preferably around 11 μm); and the second silica particles having a d₅₀ value of from 4 μm to 6 μm (preferably around 5 μm).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica; the first silica particles having a d₅₀ value of from 5 μm to 15 μm, and a d₉₀ value of from 25 μm to 35 μm; and the second silica particles having a d₅₀ value of from 1 μm to 9 μm, and a d₉₀ value of from 3 μm to 25 μm.

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 25% (e.g. around 20%, around 21%) of the d₅₀ value of the particles of the first abrasive silica; the first silica particles having a d₅₀ value of from 10 μm to 12 μm (preferably around 11 μm) and a d₉₀ value of from 30 μm to 32 μm (preferably around 31 μm); and the second silica particles having a d₅₀ value of from 1 μm to 3 μm (preferably around 2 μm), and a d₉₀ value of from 3 μm to 5 μm (preferably around 4 μm).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 25% to 35% (e.g. around 32%, around 33%, around 34%) of the d₅₀ value of the particles of the first abrasive silica; the first silica particles having a d₅₀ value of from 10 μm to 12 μm (preferably around 11 μm), and a d₉₀ value of from 30 μm to 32 μm (preferably around 31 μm); and the second silica particles having a d₅₀ value of from 3 μm to 5 μm (preferably around 4 μm), and a d₉₀ value of from 6 μm to 8 μm (preferably around 7 μm).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 40% to 50% (e.g. around 43, around 44, around 45) of the d₅₀ value of the particles of the first abrasive silica; the first silica particles having a d₅₀ value of from 10 μm to 12 μm (preferably around 11 μm), and a d₉₀ value of from 30 μm to 32 μm (preferably around 31 μm); and the second silica particles having a d₅₀ value of from 4 μm to 6 μm (preferably around 5 μm), and a d₉₀ value of from 12 μm to 14 μm (preferably around 13 μm).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 25% (e.g. around 21%, around 22%) of the d₅₀ value of the particles of the first abrasive silica; the first silica particles having a d₅₀ value of from 10 μm to 12 μm (preferably around 11 μm) and a d₉₀ value of from 28 μm to 30 μm (preferably around 29 μm); and the second silica particles having a d₅₀ value of from 1 μm to 3 μm (preferably around 2 μm), and a d₉₀ value of from 3 μm to 5 μm (preferably around 4 μm).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 30% to 40% (e.g. around 35%, around 36%, around 37%) of the d₅₀ value of the particles of the first abrasive silica; the first silica particles having a d₅₀ value of from 10 μm to 12 μm (preferably around 11 μm), and a d₉₀ value of from 28 μm to 30 μm (preferably around 29 μm); and the second silica particles having a d₅₀ value of from 3 μm to 5 μm (preferably around 4 μm), and a d₉₀ value of from 6 μm to 8 μm (preferably around 7 μm).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 40% to 55% (e.g. around 47, around 48, around 49, around 50) of the d₅₀ value of the particles of the first abrasive silica; the first silica particles having a d₅₀ value of from 10 μm to 12 μm (preferably around 11 μm), and a d₉₀ value of from 28 μm to 30 μm (preferably around 29 μm); and the second silica particles having a d₅₀ value of from 4 μm to 6 μm (preferably around 5 μm), and a d₉₀ value of from 12 μm to 14 μm (preferably around 13 μm).

In some embodiments, the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica; the first silica particles having a d₅₀ value of from 5 μm to 15 μm, and a d₉₀ value of from 25 μm to 35 μm, and an oil absorption value of from 75 to 150 g/100 g; and the second silica particles having a d₅₀ value of from 1 μm to 9 μm, and a d₉₀ value of from 3 μm to 25 μm, and an oil absorption value of from 30 to 120 g/100 g.

Abrasiveness

The abrasive silica particles suitable for use in a dentifrice composition according to the aspects of invention disclosed herein (i.e. the population of abrasive silica particles as a whole) may have a Relative Dentine Abrasion (RDA) value of 150 or less, optionally 120 or less, optionally still 100 or less. The abrasive silica particles may have a RDA value of 30 or more, optionally at 40 or more, and preferably an RDA of 45 or more. In embodiments, the abrasive silica particles according to the present invention may have an RDA of from 30 to 150, optionally from 30 to 120, optionally still from 40 to 100 e.g. from 45 to 80.

The RDA of the first silica particles is typically less than the RDA of the second silica particles. In other words, the second silica particles typically have an RDA that is greater than the RDA of the first silica particles. The optional third silica particles, and optionally still further (e.g. fourth) silica particles also typically have a RDA that is greater than the RDA of the first silica particles.

In embodiments, the abrasive silica particles comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica, and wherein the second silica particles have a RDA that is greater than the RDA of the first silica particles.

In embodiments, the first silica particles have a Relative Dentine Abrasion (RDA) value that is from 10% to 70% of the RDA value of the second silica particles, optionally from to 50%, optionally still from 15 to 40%, such as from 15 to 35%.

In embodiments, the first silica particles have a RDA value of 110 or less, optionally 105 or less. In embodiments, the first silica particles have a RDA value of 30 or more, optionally 40 or more, and optionally still an RDA of 50 or more. In embodiments, the first silica particles have an RDA value of from 30 to 110, optionally from 40 to 80, optionally still from 50 to 60 (e.g. 50-55).

In embodiments, the second silica particles have an RDA value of 350 or less, preferably 300 or less, more preferably 250 or less. In embodiments, the second silica particles have an RDA value of 200 or less. The second silica particles may have an RDA value of 120 or more, optionally 130 or more, and preferably an RDA of 140 or more. In embodiments, the second silica particles have an RDA value of from 120 to 300, optionally from 130 to 290, optionally still from 140 to 280. The second silica particles may for example have an RDA of from 150 to 180.

If present in the composition, the optional particles of a third abrasive silica, and optionally still further (e.g. fourth) abrasive silica, may have a RDA value in accordance with the RDA values outlined above in respect of the second abrasive silica.

In embodiments, the abrasive silica particles comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica, the particles of the first abrasive silica having an RDA of from 30 to 110, and the particles of the second abrasive silica having an RDA of from 120 to 300.

In embodiments, the abrasive silica particles comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica, the particles of the first abrasive silica having an RDA of from 30 to 60, optionally from 45 to 55 (e.g. around 51, around 52, around 53, around 54), and the particles of the second abrasive silica having an RDA of from 120 to 300. The RDA of the second abrasive silica may optionally be from 130 to 170, optionally still from 140 to 160, such as from 145 to 155 (e.g. around 153, around 154). The RDA of the second abrasive silica may optionally be from 150 to 190, optionally still from 160 to 180, such as from 165 to 175 (e.g. around 170, around 171, around 172, around 173). The RDA of the second abrasive silica may optionally be from 250 to 290, optionally still from 260 to 280, such as from 265 to 275 (e.g. around 270, around 271, around 272, around 273).

In embodiments, the abrasive silica particles comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica, the particles of the first abrasive silica having an RDA of from 90 to 120, optionally from 100 to 110 (e.g. around 104, around 105, around 106), and the particles of the second abrasive silica having an RDA of from 120 to 300. The RDA of the second abrasive silica may optionally be from 130 to 170, optionally still from 140 to 160, such as from 145 to 155 (e.g. around 153, around 154). The RDA of the second abrasive silica may optionally be from 150 to 190, optionally still from 160 to 180, such as from 165 to 175 (e.g. around 170, around 171, around 172, around 173). The RDA of the second abrasive silica may optionally be from 250 to 290, optionally still from 260 to 280, such as from 265 to 275 (e.g. around 270, around 271, around 272, around 273).

In embodiments, the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica; the first silica particles having a d₅₀ value of from 5 μm to 15 μm, and a d₉₀ value of from 25 μm to 35 μm, and an oil absorption value of from 75 to 150 g/100 g, and a RDA value of from 30 to 110; and the second silica particles having a d₅₀ value of from 1 μm to 9 μm, and a d₉₀ value of from 3 μm to 25 μm, and an oil absorption value of from 30 to 120 g/100 g, and an RDA value of from 120 to 300.

The abrasive silica particles according to the present invention may have a Plastic Abrasion Value (PAV) of 15 or less, optionally 10 or less. The abrasive silica particles may have a PAV of 3 or more, optionally at 4 or more. The abrasive silica particles may have a PAV of from 3 to 15, optionally 3 to 10, optionally still 4 to 15 (e.g. 4-10).

In embodiments, the abrasive silica particles according to the present invention have a PAV of from 2 to 6, optionally from 3 to 5 (e.g. around 4).

In embodiments, the abrasive silica particles according to the present invention have a PAV of from 4 to 10, optionally from 5 to 10 (e.g. around 7, around 8).

In embodiments, the abrasive silica particles according to the present invention have a PAV of from 6 to 15, optionally from 7 to 14 (e.g. around 10).

In embodiments, the abrasive silica particles according to the present invention have a PAV of from 6 to 15, optionally from 7 to 14 (e.g. around 10, around 12, around 14).

In embodiments, the abrasive silica particles comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica, and wherein the second silica particles have a PAV that is greater than the PAV of the first silica particles.

The PAV of the first silica particles is generally less than the PAV of the second silica particles. In other words, the second silica particles generally have a PAV that is greater than the PAV of the first silica particles. The optional third silica particles, and optionally still further (e.g. fourth) silica particles generally have a PAV that is greater than the PAV of the first silica particles.

In embodiments, the first silica particles have a Plastic Abrasion Value (PAV) that is from 5% to 80% of the PAV value of the second silica particles, optionally from 7 to 50%, such as from 9 to 45%.

In embodiments, the first silica particles have a PAV of 8 or less, optionally 7 or less, optionally still 5 or less. In embodiments, the first silica particles have a PAV of 2 or more, optionally 3 or more. In embodiments, the first silica particles have a PAV of from 2 to 8, optionally from 3 to 7, optionally still from 3 to 5, such as 3 to 4, wherein the PAV of the first silica particles is less than the PAV of the second silica particles.

In embodiments, the second silica particles have a PAV of 50 or less, optionally 40 or less. In embodiments, the second silica particles have a PAV of 7 or more, optionally 8 or more. In embodiments, the second silica particles have a PAV of from 7 to 50, optionally from 8 to 40, wherein the PAV of the second silica particles is greater than the PAV of the first silica particles.

The optional particles of a third abrasive silica, and optionally still further (e.g. fourth) abrasive silica, may have a PAV value in accordance with the PAV value outlined above in respect of the second abrasive silica.

In embodiments, the abrasive silica particles comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica, the particles of the first abrasive silica having a PAV of from 2 to 5, optionally from 3 to 5 (e.g. around 4), and the particles of the second abrasive silica having a PAV of from 7 to 50. The PAV of the second abrasive silica may optionally be from 7 to 10, optionally still from 8 to 9. The PAV of the second abrasive silica may optionally be from 15 to 25, optionally still from 18 to 23 (e.g. around 19, around 20, around 21, around 22). The PAV of the second abrasive silica may optionally be from 25 to 50, optionally still from 35 to 40, (e.g. around 35, around 36, around 37).

In embodiments, the abrasive silica particles comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica, the particles of the first abrasive silica having a PAV of from 4 to 8, optionally from 5 to 7 (e.g. around 6), and the particles of the second abrasive silica having a PAV of from 7 to 50. The PAV of the second abrasive silica may optionally be from 7 to 10, optionally still from 8 to 9. The PAV of the second abrasive silica may optionally be from 15 to 25, optionally still from 18 to 23 (e.g. around 19, around 20, around 21, around 22). The PAV of the second abrasive silica may optionally be from 25 to 50, optionally still from 35 to 40, (e.g. around 35, around 36, around 37).

In embodiments, the abrasive silica particles according to the present invention comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica; the first silica particles having a d₅₀ value of from 5 μm to 15 μm, and a d₉₀ value of from 25 μm to 35 μm, and an oil absorption value of from 75 to 150 g/100 g, and a PAV value of from 2 to 8; and the second silica particles having a d₅₀ value of from 1 μm to 9 μm, and a d₉₀ value of from 3 μm to 25 μm, and an oil absorption value of from 30 to 120 g/100 g, and a PAV value of from 7 to 50, wherein the PAV of the second silica particles is greater than the PAV of the first silica particles.

Oil Absorption Value/Porosity

The oil absorption value of particles of an abrasive silica is correlated with the porosity of a given silica abrasive silica. Typically, the abrasive silicas according to the present invention are porous silicas, e.g. precipitated silicas.

In embodiments, the abrasive silica particles (i.e. the population of abrasive silica particles) according to the present invention have an oil absorption value of 150 g/100 g or less, optionally 140 g/100 g or less. The abrasive silica particles according to the first aspect of the present invention may have an oil absorption value of 75 g/100 g or more, optionally 80 g/100 g or more, optionally 100 g/100 g or more. For example, the abrasive silica particles according to the first aspect of the present invention may have an oil absorption off from 75 g/100 g to 150 g/100 g, optionally from 80 g/100 g to 145 g/100 g, optionally still from 90 to 140 g/100 g. In embodiments, the oil absorption value of the abrasive silica particles may be from 90 to 150 g/100 g, optionally from 110 to 135 g/100 g, optionally still from 120 to 140 g/100 g (e.g. around 130, around 135). In embodiments, the oil absorption value of the abrasive silica particles may be from 80 to 120 g/100 g, optionally from 90 to 110 g/100 g (e.g. around 100, around 101, around 102 g/100 g).

In embodiments, the abrasive silica particles comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica, and wherein the second silica particles have an oil absorption value that is less than the oil absorption value of the second silica particles.

In typical embodiments of the invention, the oil absorption value of the first silica particles is greater than the oil absorption value of the second silica particles. In other words, the second silica particles typically have an oil absorption value that is less than the oil absorption value of the first silica particles. If present, the optional third silica particles, and optionally still further (e.g. fourth) silica particles may each also have an oil absorption value that is less than the oil absorption value of the first silica particles.

In preferred embodiments of the invention described herein (e.g. for the first aspect of the invention), the oil absorption value of the particles of the second abrasive silica is less than the oil absorption value of the particles of the first abrasive silica, and is from 30% to 70% of the oil absorption value of the particles of the first abrasive silica.

In embodiments, the abrasive silica particles comprise particles of a first abrasive silica in an amount of at least 90 wt % relative to the total weight of the abrasive silica particles; and particles of a second abrasive silica in an amount of up to 10 wt % relative to the total weight of the abrasive silica particles. The weight median particle diameter (d₅₀) of the particles of the second abrasive silica is less than the weight median particle diameter (d₅₀) of the particles of the first abrasive silica, and is from 15% to 70% of the d₅₀ value of the particles of the first abrasive silica. The oil absorption value of the particles of second abrasive silica is less than the oil absorption value of the particles of the first abrasive silica, and is from 30% to 70% of the oil absorption value of the particles of the first abrasive silica.

In embodiments, the second silica particles have an oil absorption value that is from 35 to 65% of the oil absorption value of the first silica particles, optionally from 40 to 60%, such as from 44 to 55%.

In embodiments, the first silica particles have an oil absorption value of 150 g/100 g or less. The first silica particles may have an oil absorption value of 75 g/100 g or more, optionally 80 g/100 g or more, optionally 100 g/100 g or more. In embodiments, the first silica particles have an oil absorption value of from 75 to 150 g/100 g, optionally from 80 to 145 g/100 g, optionally still from 90 to 140 g/100 g. In embodiments, the oil absorption value of the first abrasive silica may be from 90 to 150 g/100 g, optionally from 110 to 135 g/100 g, optionally still from 120 to 140 g/100 g (e.g. around 130, around 135). In embodiments, the oil absorption value of the first abrasive silica may be from 80 to 120 g/100 g, optionally from 90 to 110 g/100 g (e.g. around 100, around 101, around 102)

In embodiments, the second silica particles have an oil absorption value of 120 g/100 g or less, optionally 110 g/100 g or less, optionally still 100 g/100 g or less, such as 85/100 g or less. In embodiments, the second silica particles have an oil absorption value of 30 or more, optionally 40 or more. In embodiments, the second silica particles have an oil absorption value of from 30 to 120, optionally from 40 to 115, optionally still from 45 to 110.

In embodiments, the second silica particles have an oil absorption value of from 50 to 100 g/100 g, optionally from 50 g/100 g to 85 g/100 g, and optionally still from 50 to 80 g/100 g. In embodiments, the oil absorption value of the second abrasive silica may be from 50 to 70 g/100 g, optionally from 55 to 65 g/100 g (e.g. around 60).

In embodiments, the oil absorption value of the second abrasive silica may be from 30 to 70 g/100 g, optionally from 40 to 60 g/100 g, optionally still from 45 to 55 g/100 g (e.g. around 46, around 47, around 48).

The optional particles of a third abrasive silica, and optionally still further (e.g. fourth) abrasive silica, may have an oil absorption value in accordance with the oil absorption values outlined above in respect of the second abrasive silica.

In embodiments, the abrasive silica particles according to the present invention comprise first silica particles having an oil absorption value of from 75 to 150 g/100 g, and the second silica particles have an oil absorption value of from 30 to 120 g/100 g.

In embodiments, the abrasive silica particles of the present invention further comprise particles of a third abrasive silica, wherein the particles of third abrasive silica has an oil absorption value as outlined above in respect of the oil absorption value of the second abrasive silica.

In embodiments, the abrasive silica particles comprise particles of a first abrasive silica in an amount of at least 90 wt % relative to the total weight of the abrasive silica particles; and particles of a second abrasive silica in an amount of up to 10 wt % relative to the total weight of the abrasive silica particles; wherein the weight median particle diameter (d₅₀) of the particles of the second abrasive silica is less than the weight median particle diameter (d₅₀) of the particles of the first abrasive silica and from 15% to 70% of the d₅₀ value of the particles of the first abrasive silica, and wherein the second silica particles have an oil absorption value that is less than the oil absorption value of the second silica particles, wherein the oil absorption value of the first abrasive silica is from 75 to 150 g/100 g, and where the oil absorption value of second abrasive silica is from 30 to 120 g/100 g.

In embodiments, the abrasive silica particles according to the invention comprise particles of a first abrasive silica in an amount of at least 90 wt % relative to the total weight of the abrasive silica particles; and particles of a second abrasive silica in an amount of up to 10 wt % relative to the total weight of the abrasive silica particles, wherein weight median particle diameter (d₅₀) of the particles of the second abrasive silica is less than the weight median particle diameter (d₅₀) of the particles of the first abrasive silica, and is from 15% to 70% of the d₅₀ value of the particles of the first abrasive silica, and wherein the oil absorption value of the particles of second abrasive silica is less than the oil absorption value of the particles of the first abrasive silica, and is from 30% to 70% of the oil absorption value of the particles of the first abrasive silica, and wherein the oil absorption value of the first abrasive silica is from 75 to 150 g/100 g, and where the oil absorption value of second abrasive silica is from 30 to 120 g/100 g.

In embodiments, the abrasive silica particles according to the invention comprise particles of a first abrasive silica in an amount of at least 90 wt % relative to the total weight of the abrasive silica particles; and particles of a second abrasive silica in an amount of up to 10 wt % relative to the total weight of the abrasive silica particles, wherein weight median particle diameter (d₅₀) of the particles of the second abrasive silica is less than the weight median particle diameter (d₅₀) of the particles of the first abrasive silica, and is from 15% to 70% of the d₅₀ value of the particles of the first abrasive silica, and wherein the oil absorption value of the particles of second abrasive silica is less than the oil absorption value of the particles of the first abrasive silica, and is from 30% to 70% of the oil absorption value of the particles of the first abrasive silica, and wherein the oil absorption value of the first abrasive silica is from 90 to 150 g/100 g, optionally from 110 to 135 g/100 g, optionally still from 120 to 140 g/100 g (e.g. around 130, around 135); and where the oil absorption value of second abrasive silica is from 30 to 120 g/100 g. The oil absorption value of the second abrasive silica may be from 30 to 70 g/100 g, optionally from 40 to 60 g/100 g, optionally still from 45 to 55 g/100 g (e.g. around 46, around 47, around 48). The oil absorption value of the second abrasive silica may be from 50 to 80 g/100 g, optionally still from 55 to 65 g/100 g (e.g. around 60).

In embodiments, the abrasive silica particles according to the invention comprise particles of a first abrasive silica in an amount of at least 90 wt % relative to the total weight of the abrasive silica particles; and particles of a second abrasive silica in an amount of up to 10 wt % relative to the total weight of the abrasive silica particles, wherein weight median particle diameter (d₅₀) of the particles of the second abrasive silica is less than the weight median particle diameter (d₅₀) of the particles of the first abrasive silica, and is from 15% to 70% of the d₅₀ value of the particles of the first abrasive silica, and wherein the oil absorption value of the particles of second abrasive silica is less than the oil absorption value of the particles of the first abrasive silica, and is from 30% to 70% of the oil absorption value of the particles of the first abrasive silica, and wherein the oil absorption value of the first abrasive silica is from 80 to 120 g/100 g, optionally from 90 to 110 g/100 g (e.g. around 100, around 101, around 102), and where the oil absorption value of second abrasive silica is from 30 to 120 g/100 g. The oil absorption value of the second abrasive silica may be from 30 to 70 g/100 g, optionally from 40 to 60 g/100 g, optionally still from 45 to 55 g/100 g (e.g. around 46, around 47, around 48). The oil absorption value of the second abrasive silica may be from 50 to 80 g/100 g, optionally from 50 to 70 g/100 g, optionally still from 55 to 65 g/100 g (e.g. around 60).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt %, optionally 2 to 7 wt %, relative to the total weight of the abrasive silica particles; wherein the weight median particle diameter (d₅₀) of the particles of the second abrasive silica is less than the weight median particle diameter (d₅₀) of the particles of the first abrasive silica and from 15% to 25% (e.g. around 20%, around 21%) of the d₅₀ value of the particles of the first abrasive silica; the first silica particles having a d₅₀ value of from 10 μm to 12 μm (preferably around 11 μm) and a d₉₀ value of from 30 μm to 32 μm (preferably around 31 μm), and an oil absorption value of from 125 to 135 g/100 g (preferably from 130 to 135 g/100 g e.g. 134 g/100 g); and the second silica particles having a d₅₀ value of from 1 μm to 3 μm (preferably around 2 μm), and a d₉₀ value of from 3 μm to 5 μm (preferably around 4 μm), and an oil absorption value of from 55 to 65 g/100 g (preferably around 60 g/100 g).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt %, %, optionally 2 to 7 wt %, relative to the total weight of the abrasive silica particles; wherein the weight median particle diameter (d₅₀) of the particles of the second abrasive silica is less than the weight median particle diameter (d₅₀) of the particles of the first abrasive silica and from 25% to 35% (e.g. around 32%, around 33%, around 34%) of the d₅₀ value of the particles of the first abrasive silica; the first silica particles having a d₅₀ value of from 10 μm to 12 μm (preferably around 11 μm), and a d₉₀ value of from 30 μm to 32 μm (preferably around 31 μm), and an oil absorption value of from 125 to 135 g/100 g (preferably from 130 to 135 g/100 g e.g. 134 g/100 g); and the second silica particles having a d₅₀ value of from 3 μm to 5 μm (preferably around 4 μm), and a d₉₀ value of from 6 μm to 8 μm (preferably around 7 μm), and an oil absorption value of from 60 to 70 g/100 g (preferably around 65 g/100 g).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt %, %, optionally 2 to 7 wt %, relative to the total weight of the abrasive silica particles; wherein the weight median particle diameter (d₅₀) of the particles of the second abrasive silica is less than the weight median particle diameter (d₅₀) of the particles of the first abrasive silica and from 40% to 50% (e.g. around 43, around 44, around 45) of the d₅₀ value of the particles of the first abrasive silica; the first silica particles having a d₅₀ value of from 10 μm to 12 μm (preferably around 11 μm), and a d₉₀ value of from 30 μm to 32 μm (preferably around 31 μm), and an oil absorption value of from 125 to 135 g/100 g (preferably from 130 to 135 g/100 g e.g. 134 g/100 g); and the second silica particles having a d₅₀ value of from 4 μm to 6 μm (preferably around 5 μm), and a d₉₀ value of from 12 μm to 14 μm (preferably around 13 μm), and an oil absorption value of from 40 to 50 g/100 g (preferably around 45 g/100 g).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt %, %, optionally 2 to 7 wt %, relative to the total weight of the abrasive silica particles; wherein the weight median particle diameter (d₅₀) of the particles of the second abrasive silica is less than the weight median particle diameter (d₅₀) of the particles of the first abrasive silica and from 15% to 25% (e.g. around 21%, around 22%) of the d₅₀ value of the particles of the first abrasive silica; the first silica particles having a d₅₀ value of from 10 μm to 12 μm (preferably around 11 μm) and a d₉₀ value of from 28 μm to 30 μm (preferably around 29 μm), and an oil absorption value of from 90 to 100 g/100 g (preferably around 95 g/100 g); and the second silica particles having a d₅₀ value of from 1 μm to 3 μm (preferably around 2 μm), and a d₉₀ value of from 3 μm to 5 μm (preferably around 4 μm), and an oil absorption value of from 55 to 65 g/100 g (preferably around 60 g/100 g).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt %,%, optionally 2 to 7 wt %, relative to the total weight of the abrasive silica particles; wherein the weight median particle diameter (d₅₀) of the particles of the second abrasive silica is less than the weight median particle diameter (d₅₀) of the particles of the first abrasive silica and from 30% to 40% (e.g. around 35%, around 36%, around 37%) of the d₅₀ value of the particles of the first abrasive silica; the first silica particles having a d₅₀ value of from 10 μm to 12 μm (preferably around 11 μm), and a d₉₀ value of from 28 μm to 30 μm (preferably around 29 μm), and an oil absorption value of from 90 to 100 g/100 g (preferably around 95 g/100 g); and the second silica particles having a d₅₀ value of from 3 μm to 5 μm (preferably around 4 μm), and a d₉₀ value of from 6 μm to 8 μm (preferably around 7 μm), and an oil absorption value of from 60 to 70 g/100 g (preferably around 65 g/100 g).

In some embodiments the abrasive silica particles according to the present invention comprise particles of a first abrasive silica in an amount of at least 90 wt % relative to the total weight of the abrasive silica particles; and particles of a second abrasive silica in an amount of up to 10 wt %%, optionally 2 to 7 wt %, relative to the total weight of the abrasive silica particles; wherein the weight median particle diameter (d₅₀) of the particles of the second abrasive silica is less than the weight median particle diameter (d₅₀) of the particles of the first abrasive silica and from 40% to 55% (e.g. around 47, around 48, around 49, around 50) of the d₅₀ value of the particles of the first abrasive silica; the first silica particles having a d₅₀ value of from 10 μm to 12 μm (preferably around 11 μm), and a d₉₀ value of from 28 μm to 30 μm (preferably around 29 μm), and an oil absorption value of from 90 to 100 g/100 g (preferably around 95 g/100 g); and the second silica particles having a d₅₀ value of from 4 μm to 6 μm (preferably around 5 μm), and a d₉₀ value of from 12 μm to 14 μm (preferably around 13 μm), and an oil absorption value of from 40 to 50 g/100 g (preferably around 45 g/100 g).

Cleaning Performance (FT₁₀₀)

The abrasive silica particles (i.e. the population of abrasive silica particles as a whole) according to the invention may have a ferric tannate cleaning value at 100 strokes (FT₁₀₀) of from 40 to 100, such as from 50 to 95, optionally from 60 to 95, optionally still from 65 to 90. The abrasive silica particles may have a FT₁₀₀ of from 60 to 80, optionally from 60 to 70. The abrasive silica particles may have a FT₁₀₀ of from 70 to 90, optionally from 70 to 80. The abrasive silica particles may have a FT₁₀₀ of from 75 to 95, optionally from 80 to 90. The FT₁₀₀ values are obtained from a preparation of the abrasive silica particles as a slurry, in accordance with the Ferric Tannate (FT) cleaning protocol described herein.

Compositions Containing Silica Particles of the Invention

The present invention additionally provides a composition for use in a dentifrice, the composition comprising abrasive silica particles in accordance with the present invention (e.g. the first aspect of the present invention). The composition may comprise from 1 to 99 wt % of the abrasive silica particles, optionally from 20 to 80 wt %, optionally still from 40 to 60 wt % of the abrasive silica particles.

The composition may further comprise a carrier, e.g. a fluid carrier, such as a liquid carrier, powder, or the like. Powder compositions containing the inventive abrasive silicas of the first aspect of the invention as a component of the powder are envisaged. The carrier may comprise one or more of water, solvents, sugars/sweeteners (e.g. sorbitol, glycerol, xylitol and combinations thereof), surfactants (e.g. sodium lauryl sulfate), humectants (e.g. polyethylene glycol), titanium dioxide, gums (e.g. xanthan), salts (e.g. fluoride salts such as sodium fluoride), thickeners, and combinations thereof. The carrier may be present in the composition in an amount from 0.1 to 90 wt %, optionally 10 to 80 wt %, optionally still 20 to 70 wt %.

In embodiments, the composition may comprise silica particles not present in the abrasive silica particles according to the present invention (i.e. silica particles that are not particles as defined for the first, second, third or further (e.g. fourth) abrasive silicas). Such silica particles may for instance be non-abrasive silica particles. Such silica particles may have an RDA of less than 30, typically less than 20, and optionally less than 10 (e.g. around 8). Such silica particles may for example be a thickener, for example SORBOSIL® TC15, commercially available from PQ Silicas UK Limited. If silica particles not according to the first, second, third or further (e.g. fourth) abrasive silicas of the invention are present in the composition, they may typically be present in the composition in an amount from 5 to 30 wt %, optionally 10 to 20 wt % (e.g. 11 wt %, 15 wt %, 19 wt %).

In embodiments, the composition according to the present invention further comprises one or more surfactants. Surfactants may include water-soluble salts. Suitable surfactants may be selected from anionic surfactants (e.g. sodium lauryl sulfate, sodium dodecyl benzene sulfonate, sodium lauryl sulfoacetate, 1,2-dihydroxypropane sulfonate, and the like), cationic surfactants (e.g. betaines), and combinations thereof. The surfactant may be present in the composition in an amount from 0.1 to 10 wt %, optionally 1 to 5 wt %, optionally still from 2 to 3 wt %.

Compositions in accordance with the present addition may comprise one or more polyols. Suitable polyols may be selected from sorbitol, glycol, propylene glycol, polyethylene glycol (PEG), and combinations thereof.

The compositions in accordance with the present invention may contain additional excipients, colourants, flavourants, carrageenan (rich oss), sodium carboxymethyl cellulose, starch, polyvinyl pyrollidone, hydroxyethyl propyl cellulose, hydroxybutyl methyl cellulose, hydroxypropyl methyl cellulose, hydroxyethyl cellulose, and combinations thereof.

Dentifrices

The present invention provides a dentifrice comprising the abrasive silica particles according the present invention, or the composition according to the invention. The dentifrice may be a powder, paste, or gel. Preferably, the dentifrice is a paste.

In embodiments, the dentifrice comprises from 0.1 to 50 wt % of the abrasive silica particles according to the present invention, optionally from 1 to 30 wt %, and optionally still from 10 to 25 wt % (e.g. 20 wt %).

In embodiments, the RDA of the dentifrice is 250 or less, optionally 200 or less. The RDA of the dentifrice may be 5 or more. The RDA of the dentifrice may be from 5 to 200, optionally 40 to 150, optionally still from 70 to 120.

The dentifrice may be prepared by combining silica particles according to the first aspect of the invention, or a composition according to the second aspect of the invention, with one of more excipients suitable for dentifrice applications.

Method of Preparing Abrasive Silica Particles

The present invention further provides a method of preparing abrasive silica particles for use in a dentifrice composition. The method comprises combining particles of a first abrasive silica and particles of a second abrasive silica to provide the abrasive silica particles. The particles of the first abrasive silica are present in an amount of at least 90 wt % relative to the total weight of the abrasive silica particles, and the particles of the second abrasive silica are present in an amount of up to 10 wt % relative to the total weight of the abrasive silica particles. The weight median particle diameter (d₅₀) of the second silica particles is less than the first silica particles, and is from 15% to 70% of the d₅₀ value of the first silica particles.

In embodiments, the combining step comprises combining particles of the first abrasive silica and the second abrasive silica and also particles of a third abrasive silica, to provide the abrasive silica particles. The step may optionally comprise combining particles of further abrasive silicas (e.g. particles of a fourth abrasive silica). The weight median particle diameter d₅₀ of the particles of the third silica is less than the first silica particles and from 15% to 70% of the weight median particle diameter d₅₀ of the first silica particles. The combined weight of the particles of the second and third silicas does not exceed 10 wt % relative to the total weight of the abrasive silica particles.

In embodiments, the combining comprises mixing the silica particles, optionally wherein the mixing provides a homogeneous mixture of the silica particles.

The skilled person will be familiar with methods for mixing the first ‘bulk’ abrasive silica and the second abrasive silica. Suitable apparatus for mixing may include a powder blender mixer (e.g. a Turbula® mixer). The first and second abrasive silicas may be mixed simultaneously (i.e. added to the mixture in equal quantities until fully mixed), or added sequentially, in any order. The first abrasive silica may be added (e.g. gradually) to the second abrasive silica, or the second abrasive silica may be added (e.g. gradually) to the first abrasive silica.

In embodiments, the method is a method of preparing abrasive silica particles as defined in accordance with the present invention.

The method may further comprise the additional steps of contacting the abrasive silica particles obtained by the above method steps with one or more excipients suitable for dentifrice applications, to form a dentifrice. The dentifrice so prepared may be as defined according to the third aspect of the invention, e.g. any dentifrice as defined herein.

The present invention further provides abrasive silica particles prepared according to the method of preparing abrasive silica particles as defined in accordance with the present invention for use in a dentifrice composition. Such particles may be as defined herein for the first aspect of the invention.

General Methods

Abrasive Silicas

The skilled person will be readily able to select suitable silicas for use as particles of first, second, or third (or further) abrasive silicas as described herein from the large variety of commercial silicas available. Methods for the preparation of suitable particles of first, second, or third (or further) abrasive silicas for use in accordance with the present invention will also be familiar to those skilled in the art. For example, methods of preparing abrasive silicas are described in “The Chemistry of Silica” by Ralph K. Iler (ISBN: 9780471024040). Particles of precipitated abrasive silicas suitable for use in the present invention may be prepared by providing an alkaline metal silicate solution, mixing the solution with acid, optionally in the presence of an electrolyte, stirring and filtering out the precipitated silica. The resulting precipitate filter cake is then washed, dried, and comminuted to the desired particle size.

European patent EP1976482 describes the preparation of suitable silicas for use in accordance with the present invention. Examples 1C, 1D, 1E and 4A described in European patent EP1976482 are particularly suitable for use in accordance with the present invention, particularly as particles of a second (or third or further) abrasive silica.

Abrasive silicas described in U.S. Pat. No. 5,098,695, EP0835223 and EP0785169 are particularly suitable for use in accordance with the present invention, particularly as particles of a first “bulk” abrasive silica.

Particular examples of suitable particles of a first “bulk” abrasive silica include SORBOSIL® AC39 and SORBOSIL® AC36 commercially available from PQ Silicas UK Limited. Other suitable commercially available first (i.e. “bulk”) abrasive silicas that may be used in accordance with the present invention include Tixosil® 123 commercially available from Solvay, Zeodent® 113 and Zeodent® 116 commercially available from Evonik, and Sylodent® VP5 commercially available from Grace.

General methods by which silicas for use in accordance with the present invention may be prepared are outlined below with reference to U.S. Pat. No. 5,447,704 and European Patent EP0308165.

U.S. Pat. No. 5,447,704 describes a suitable method of preparing a suitable amorphous precipitated silica produced by the reaction of sodium silicate, having a silica:Na₂O ratio in the range from 1.8:1 to 3.5:1, with mineral acid, with the concentration and volume of the reactants controlled to give a reaction in the pH range from about 10 to about 10.5, in the presence of a water soluble electrolyte comprising a cation selected from the group comprising aluminium, magnesium, calcium, sodium and potassium with an associated anion selected from the group comprising bromide, carbonate, chloride, nitrate, acetate and sulphate wherein the electrolyte:silica weight ratio is from about 0.1:1 to about 2:1, the precipitation reaction being performed in the temperature range of about 95° C. to about 100° C.

European Patent EP0308165 describes a method of preparing suitable amorphous abrasive silicas produced by the reaction of sodium silicate, having a silica:Na₂O ratio in the range from 3.2:1 to 3.4:1, with mineral acid, with the concentration and volume of the reactants controlled to give a reaction in the pH range from about 10 to about 10.5, in the presence of a water soluble electrolyte comprising a cation selected from sodium and potassium with an associated anion selected from chloride and sulphate wherein the electrolyte:silica weight ratio is from about 0.4:1 to about 1.2:1, the precipitation reaction being performed in the temperature range of about 45° C. to about 55° C., the pH of the reaction medium then being made acidic by addition of a mineral acid, separating and washing the resultant silica product.

Once silicas have been prepared (for example according to the methods of U.S. Pat. No. 5,447,704 or European Patent EP 0308165 outlined above), a mechanical mill (e.g. a hammer mill) may be used to comminute the abrasive silicas to a desired particle size. Mechanical milling generally yields weight median particle diameters (d₅₀) of from 7 to 20 μm. For smaller particle sizes, a high-energy comminution processes may be used e.g. micronisation. Micronisation may be achieved, for example, using one or more of a jet, fluid energy mill, a pancake microniser, fluidised bed micronisation, and opposed jet micronisers.

Optionally, the material may be subjected to classification, screening or sieving at any stage of the process in order to optimize the process and to remove excess large particles such that the preferred particle size distributions of the abrasive silicas may be obtained.

Preparation of Abrasive Silica Particles According to the Invention

Particles of first “bulk” abrasive silica (silicas A1, A2, A3) and particles of second abrasive silica (silicas B1, B2, B3, B4), and optionally particles of a third abrasive silica (from silicas B1, B2, B3, B4) were combined (e.g. in the amounts (wt %) indicated in Table 2 below) to provide a mixture. The abrasive silicas were weighed under atmospheric pressure and room temperature. The mixture was blended to provide a blended, homogeneous mixture of abrasive silica particles.

Preparation of Compositions Comprising Abrasive Silica Particles

The batches of abrasive silica particles prepared as described immediately above were added to one or more additional components (generally a carrier) and mixed to provide the composition.

Preparation of Dentifrice comprising Abrasive Silica Particles

Dentifrices comprising abrasive silica particles (of the invention or reference examples) were prepared having the following general ingredients:

| Ingredient Name | % w/w |
|---|---|
| Purified Water | 26.2 |
| Sorbitol, 70 wt % Aqueous | 39.5 |
| Solution (Non-Crystallising) | |
| Abrasive Silica Particles | 20.0 |
| Silica, Thickener Type (TC15 PR18801) | 5.50 |
| Sodium Lauryl Sulphate | 1.50 |
| Sodium Carboxymethylcellulose | 0.7 |
| (SCMC) 9M31XF | |
| PEG400 | 4.0 |
| Sodium Fluoride | 0.3 |
| Saccharin Sodium | 0.3 |
| Flavour Oil | 1.00 |
| Titanium dioxide | 1 |

The water and sorbitol were combined, and sodium fluoride and sodium saccharide were subsequently added. The resulting composition was mixed for 30 mins at ambient temperature, followed by addition of the SLS powder and further mixing for 30 mins. The flavour oil was subsequently added followed by mixing for a further 30 mins, followed by the TiO₂ and further mixing for 30 mins. To the resulting mixture, a separate mixture of SCMC and PEG400 was added, followed by mixing for 30 mins. The abrasive silica particles (i.e. of the invention, or abrasive reference silicas) were then added to the resulting mixture, followed by mixing for 30 mins. The TC15 thickener silica was added to the resulting mixture, followed by stirring for 30 mins, to provide the dentifrice. Dentifrices were provided wherein the abrasive silica particle component was either a) according to aspects of the invention (to provide dentifrices according to the invention) or b) a reference abrasive silica not according to the invention (typically containing only a single silica corresponding to one of the silicas used in the inventive compositions.

Particle Sizing

For any given batch of silica particles described herein, e.g. abrasive silica particles of the invention as a whole, or the first or second silicas used as sub-populations within the abrasive silica particles of the invention, the weight median particle diameter (d₅₀) of abrasive silica particles were determined by laser diffraction using a Malvern Mastersizer 2000 and a Hydro 2000 AG dispersion unit. Mie theory was used to calculate particle size distributions. The real value of the silica refractive index was assigned a value of 1.46 and the imaginary refractive index of the particle was assigned a value of 1.0, with water dispersant having a real refractive index of 1.33. Abrasive silica particles were dispersed ultrasonically using the Hydro 2000 AG dispersion unit at 50% power for 5 minutes in de-ionised water to form an aqueous suspension. Laser light was passed through a flow cell containing the particles dispersed in de-ionised water. The scattered light intensity was measured as a function of angle and the data used to calculate particle size distribution. Weight-based particle size measures were used, assuming constant density of the particles.

Oil Absorption Value

For any given batch of silica particles described herein, e.g. abrasive silica particles of the invention as a whole, or the first or second silicas used as sub-populations within the abrasive silica particles of the invention, the oil absorption (O/A) value was determined by the ASTM spatula rub-out method (American Society of Test Material Standards D 281). Linseed oil and abrasive silica particles were mixed by rubbing with a spatula on a smooth surface until a stiff putty-like paste was formed. The volume of oil absorbed is expressed in cm³ per 100 g of silica and is then calculated as Oil absorption value=(cm³ oil absorption×100)/(weight of silica in grams). This quantity may also be expressed in grams of oil per 100 g of silica (g/100 g) by multiplying by an assumed density of linseed oil of 0.93 grams per cm³. All oil absorption results presented herein were calculated in this fashion and are expressed in grams of oil per 100 g of silica.

Plastic Abrasion Value (PAV)

For any given batch of silica particles described herein, e.g. the abrasive silica particles of the invention as a whole, or the first or second silicas used as sub-populations within the abrasive silica particles of the invention, the Plastic Abrasion Value (PAV) test was used to measure the abrasiveness of abrasive silica particles as described herein. Perspex® has a similar hardness to dentine. Samples were prepared as a slurry as follows by mixing the following components to form a suspension:

| | |
|---|---|
| Abrasive silica particles | 2.5 grams |
| Glycerol | 10.0 grams |
| Sorbitol Syrup (70 wt % | 23.0 grams |
| sorbitol and 30 wt % water) | |

Standard clear Perspex® (grade 000; manufactured by Lucite International UK Ltd) was used. A Wet Paint Scrub Tester produced by Sheen Instruments, modified to provide a holder with a toothbrush (instead of a paintbrush). A weight of 400 g was attached to the brush assembly (which weighs 145 g) to urge the brush onto the Perspex® sheet. The toothbrush was a multi-tufted, nylon head and medium texture (e.g. a Professional Mentadent® P gum health design, or an equivalent toothbrush). A Byk Microgloss 45° detector was calibrated using a standard (56.8% gloss) reflecting plate. The gloss of a fresh Perspex® sheet was then measured, and fitted into a holder. 2 mL of the sample was placed on the sheet, and contacted with the brush head for 300 strokes. The Perspex® sheet was removed from the holder, washed, dried, and measured again. The abrasion value was determined as the difference between measured the gloss value before and after abrasion with the sample. A reference example, gave the following results:

| | Weight median | |
|---|---|---|
| | particle size | |
| Abrasive | d₅₀ (μm) | PAV |
| Calcium Carbonate | 15 | 32 |
| Silica xerogel; prepared by | 10 | 25 |
| method of UK 1264292 | | |
| Alumina trihydrate (Gibbsite) | 15 | 16 |
| Calcium Pyrophosphate | 10 | 14 |
| Dicalcium diphosphate dihydrate | 15 | 7 |

Relative Dentine Abrasion

For any given batch of silica particles described herein, e.g. the abrasive silica particles of the invention taken as a whole, or the first or second silicas used as sub-populations within the abrasive silica particles of the invention, the Relative Dentine Abrasion Test (RDA, also known as Radioactive Dentine Abrasion) was used to measure the abrasiveness of abrasive silica particles according to the present invention. The procedure of the American Dental Association (Journal of Dental Research 55 (4) page 563-573, 1976) was used, wherein extracted human teeth are irradiated with a neutron flux and subjected to a standard brushing regime. The radioactive phosphorous 32 removed from the dentine in the roots is used as the index of the abrasion of the powder or oral composition tested. A reference slurry containing 10 g of calcium pyrophosphate in 50 cm³ of 0.5% aqueous solution of sodium carboxymethyl cellulose was assigned an RDA of 100. Slurry samples comprising the abrasive silica particles according to the invention were prepared at the same wt % concentration as calcium pyrophosphate in the reference slurry. Slurry samples comprising dentifrices according to the present invention were prepared by mixing 25 g dentifrice and 40 cm³ of water to provide a slurry having the required concentration. RDA tests were performed at the Oral Health Research Institute, School of Dentistry, Indiana University, USA. The RDA of comparative dentifrices prepared according to the methods outlined above were determined using the same method.

Ferric Tannate (FT) Cleaning Test

The Ferric Tannate Cleaning tests were performed in accordance with the method described in “Dental stain prevention by abrasive toothpastes: A new in vitro test and its correlation with clinical observations”, P. L. Dawson et al., J. Cosmet. Sci., 49, 275-283 (1998). The test substrate was a pure hydroxyapatite (HAP) disc was polished using a Buehler rotary grinder and P600 wet paper, followed by P1200 lapping paper. The whiteness of the discs (using the CIE 1976 L*a*b* system) before cleaning, L* (clean), was then measured using a Minolta Chroma-meter CR200, which has been calibrated against a standard calibration tile. The substrate was repeatedly stained with a staining solution (50 g of a 0.5% by weight solution of tannic acid and 50 g of a 0.5% by weight solution of ammonium ferric sulphate to form a fresh colloidal iron (III) tannic acid complex (“ferric tannate”)) until a darkness measurement of L*=50+/−5 as determined using a Minolta Chroma-meter CR200 was achieved. This value is designated L* (soiled). The stained substrate was mounted in a vessel containing a sample FT slurry and weighted (263 g) Mentadent® P Professional soft-nylon flat trim toothbrush heads were oscillated (150 cycles per minute) over the stained substrate surfaces using a mechanical scrubbing machine (modified Martindale Mk111 abrasion tester). Stain removal after a desired number of oscillations (e.g. 50, 100, 150, 300) was measured using a Minolta Chroma-meter CR200, corresponding to an FT_{XX} removal test result, wherein XX is the desired number of oscillations. The whiteness of the substrate after cleaning after a desired number of oscillations is L* (cleaned). Comparative abrasive performance is taken to be the percentage clean or removed at XX oscillations (e.g. 100).

For example, FT₁₀₀ is defined as the % FT₁₀₀ Removal where:

$$\% {FT}_{100} Removal = \frac{( {L}^{*} ( cleaned ) - {L}^{*} ( soiled ) )}{( {L}^{*} ( clean ) - {L}^{*} ( soiled ) )} \times 100$$

Sample FT slurries (comprising abrasive silica particles or dentifrice according to the present invention) were prepared as follows.

Preparation of FT Slurry comprising dentifrice preparation—25 g of dentifrice prepared in accordance with the method outlined above (comprising 20 wt % of abrasive silica particles according to the present invention) was combined with 50 grams of demineralised water, and the resulting mixture mixed to provide a homogeneous slurry. Comparative FT slurries comprising comparative dentifrices prepared in accordance with the method outlined above were prepared using the same method.

Preparation of FT Slurry comprising abrasive silica particles—A diluent (0.35 wt % xanthan gum, 0.5 wt % sodium lauryl sulphate, 99.15 wt % demineralised water) was added to the abrasive silica particles, and mixed to provide a homogeneous mixture. The wt % of abrasive silica particles was 3.3 wt % relative to the total final weight of the slurry. Although 3.3 wt % was used for the examples described herein, those skilled in the art will appreciate that other wt % loadings may be suitable. Comparative FT slurries comprising only particles of a single silica (i.e. a single silica corresponding to one of the particles [i.e. particles of first or second or third abrasive silica] comprised in the abrasive silica particles according to the invention) were prepared using the same method.

Pellicle Cleaning Ratio (PCR)

Pellicle cleaning ratio (PCR) methods are known to those skilled in the art. For example, suitable methods are described in to J. Dent. Res., 61:1236, 1982. Enamel surfaces (10×10 mm) to be cleaned in accordance with the methods were stained with a solution comprising PYG (peptone yeast glucose) broth, tea, coffee, mucin, FeCl₃ and Micrococcus luteus, until a uniform stain film was provided on the enamel surface. The stain film was graded photometrically using a spectrophotometer (Minolta CM2600d), and stained enamel surfaces with a stain film scoring between 30 and 42 were selected for PCR testing.

A reference slurry was prepared by mixing 10 g of Ca₂P₂O₇ with 50 mL of an aqueous glycerine solution (10 wt %) comprising 0.5% carboxymethylcellulose (CMC) (Density: 1.03 g/L). Each slurry sample to be tested was mounted on a mechanical V-8 cross-brushing machine equipped with soft nylon-filament (Oral-B 40) toothbrushes. Tension on the enamel surface was adjusted to 150 g. Specimens were brushed for 800 strokes (for approximately 4½ minutes), which is a typical number of strokes for PCR. Slurry samples were also brushed for modified numbers of brush strokes (mPCR) as indicated (e.g. 60, 120, 360, 1200). The brush strokes indicated (e.g. 60, 120, 360, 800, 1200) represent the number of brush strokes before a score was allocated. Each slurry sample was subjected to 60 strokes before being removed, scored, and then replaced. Scoring measured the difference between the pre- and post-brushing stain scores before brushing and after 60 strokes. The slurry sample was then subjected to a further 60 strokes to provide a total of 120 strokes, before being removed, scored and replaced in the same way. The same methodology was continued until the total number of cumulative strokes was 1200, after which the sample was removed and scored for a final time. The results are set out in Table 5 below.

Slurry samples comprising abrasive silica particles were prepared by mixing 10 g of abrasive silica particles with 50 mL of an aqueous glycerine (10 wt %) solution comprising 0.5 wt % CMC (Density: 1.03 g/L) to provide a slurry. Comparative slurry samples comprising only particles of a single silica (i.e. a single silica corresponding to one of the particles [i.e. particles of first or second or third abrasive silica] comprised in the abrasive silica particles according to the invention) were prepared using the same method.

Slurry samples comprising dentifrices comprising abrasive silica particles of the invention were prepared by mixing 25 grams of dentifrice with 40 mL of deionized water (1.00 g/mL) to provide dentifrice slurries. Comparative slurry samples comprising comparative dentifrices prepared in accordance with the methods outlined above were prepared using the same method.

### Examples

Particles of abrasive silicas A1, A2, A3, B1, B2, B3 and B4 used in the present examples are described in Table 1 below. Silicas A1, A2 and A3 were each respectively used as the first “bulk” abrasive silica. Silicas A1 and A2 are commercially available from PQ Silicas UK Limited, as SORBOSIL® AC39. Silica A3 is commercially available from PQ Silicas UK Limited as SORBOSIL® AC36. Other suitable commercially available first “bulk” abrasive silicas that may be used in accordance with the present invention include Tixosil® 123 commercially available from Solvay, Zeodent® 113 and Zeodent® 116 commercially available from Evonik, and Sylodent® VP5 commercially available from Grace.

Silicas B1, B2, B3 and B4 were used as particles of second abrasive silica (optionally third abrasive silica). Silica B1 was prepared in accordance with the methodology described at Example 1D of EP1976482. Silica B2 was prepared in accordance with the methodology of Example 1C of EP1976482. Silica B3 was prepared in accordance with Example 1E of EP1976482. Silica B4 was prepared in accordance with Example 6 of EP0535943. Water content (H₂O [wt %]) of the particles of abrasive silicas A1, A2, A3, B1, B2, B3 and B4 was calculated based on weight loss following heat treatment in an oven at 105° C. for 2 hours.

TABLE 1
Properties of exemplary particles of abrasive silicas

| | Particle Size (μm) | | | Oil Absorption | | | H₂O |
|---|---|---|---|---|---|---|---|
| Silica | d10 | d50 | d90 | (g/100 g) | PAV | RDA | (wt %) |
| A | | | | | | | |
| A1 | 3.33 | 11.18 | 30.68 | 130 | 3.8 | 53 | 2.3 |
| A2 | 3.30 | 10.74 | 31.85 | 134 | 3.4 | 52 | 3.0 |
| A3 | 2.25 | 10.45 | 28.58 | 96 | 6.5 | 104 | 5.9 |
| B | | | | | | | |
| B1 | 1.18 | 2.28 | 3.91 | 60 | 8.5 | 154 | 7.8 |
| B2 | 1.49 | 3.71 | 7.35 | 65 | 20.9 | 173 | 2.6 |
| B3 | 1.49 | 4.95 | 12.66 | 47 | 36.7 | 272 | 3.7 |
| B4 | 2.47 | 8.31 | 19.70 | 102 | 18.5 | 153 | 5.0 |

Abrasive silica particles were prepared in accordance with the method outlined above to provide abrasive silica particle Examples 1-31 (Table 2). Examples 4R, 10R, 11R, 16R, 20R, 21R, 22R, 23R, 24R, and 29R are reference examples. Example 21R is provided as a reference example comprising two ‘first’ abrasive silicas, A1 and A3. Example 25 comprises a third abrasive silica. The notation ‘nd’ means ‘not determined’.

TABLE 2
Examples of abrasive silica particles and properties thereof

| | | | | Relative Properties | | | | | | | | |
|---|---|---|---|---|---|---|---|---|---|---|---|---|
| | | | | Second/First Silica | | | | | | Oil | | |
| | Abrasive Silica Particles | | | | | Oil | | | | Abs | | |
| | First | Second | Third | d50 | d90 | abs | Particle Size (μm) | | | (g/100 | | |
| Ex. | (wt %) | (wt %) | (wt %) | (%) | (%) | (%) | d10 | d50 | d90 | g) | PAV | RDA |
| 1 | A1 (98) | B1 (2) | — | 20 | 13 | 46 | 3.23 | 11.10 | 31.52 | 122 | 4.2 | nd |
| 2 | A1 (97) | B1 (3) | — | 20 | 13 | 46 | 3.22 | 11.11 | 31.24 | 120 | 4.4 | 68 |
| 3 | A1 (96) | B1 (4) | — | 20 | 13 | 46 | 3.15 | 10.95 | 31.38 | 118 | 4.7 | nd |
| 4R | A1 (80) | B1 (20) | — | 20 | 13 | 46 | 2.54 | 10.51 | 33.98 | 110 | 5.9 | 60 |
| 5 | A2 (99) | B1 (1) | — | 21 | 12 | 45 | 3.30 | 10.83 | 32.39 | 131 | 4.1 | 55 |
| 6 | A2 (97) | B1 (3) | — | 21 | 12 | 45 | 3.20 | 10.70 | 32.14 | 125 | 4.2 | 60 |
| 7 | A2 (95) | B1 (5) | — | 21 | 12 | 45 | 3.17 | 10.69 | 32.07 | 124 | 4.4 | 67 |
| 8 | A2 (93) | B1 (7) | — | 21 | 12 | 45 | 3.10 | 10.77 | 32.87 | 122 | 4.4 | 70 |
| 9 | A2 (90) | B1 (10) | — | 21 | 12 | 45 | 2.96 | 10.30 | 31.39 | 116 | 4.7 | 78 |
| 10R | A2 (87) | B1 (13) | — | 21 | 12 | 45 | 2.93 | 10.37 | 31.73 | 114 | 5.3 | 84 |
| 11R | A2 (84) | B1 (16) | — | 21 | 12 | 45 | 2.79 | 10.19 | 31.70 | 114 | 5.6 | 88 |
| 12R | A2 (80) | B1 (20) | — | 21 | 12 | 45 | 2.87 | 10.21 | 31.29 | 113 | 5.5 | 80 |
| 13 | A1 (98) | B3 (2) | — | 44 | 41 | 36 | 3.25 | 10.68 | 31.65 | 125 | 7.2 | nd |
| 14 | A1 (97) | B3 (3) | — | 44 | 41 | 36 | 3.20 | 10.35 | 30.83 | 121 | 9.9 | nd |
| 15 | A1 (96) | B3 (4) | — | 44 | 41 | 36 | 3.17 | 10.27 | 31.02 | 117 | 13.3 | nd |
| 16R | A1 (80) | B3 (20) | — | 44 | 41 | 36 | 2.86 | 9.52 | 29.85 | 110 | 26.5 | nd |
| 17 | A1 (98) | B2 (2) | — | 33 | 24 | 50 | 3.22 | 10.41 | 31.25 | 126 | 5.2 | nd |
| 18 | A1 (97) | B2 (3) | — | 33 | 24 | 50 | 3.18 | 10.43 | 31.67 | 123 | 7.9 | nd |
| 19 | A1 (96) | B2 (4) | — | 33 | 24 | 50 | 3.18 | 10.42 | 31.37 | 120 | 9.4 | nd |
| 20R | A1 (80) | B2 (20) | — | 33 | 24 | 50 | 2.79 | 9.14 | 30.31 | 110 | 13.3 | nd |
| 21R | A1 (96) | A3 (4) | — | 93 | 93 | 74 | 3.21 | 11.37 | 34.00 | 133 | 4.2 | nd |
| 22R | A1 (80) | A3 (20) | — | 93 | 93 | 74 | 3.00 | 10.79 | 32.84 | 123 | 4.2 | nd |
| 23R | A1 (96) | B4 (4) | — | 74 | 64 | 78 | 3.23 | 11.33 | 33.49 | 132 | 3.8 | nd |
| 24R | A1 (80) | B4 (20) | — | 74 | 64 | 78 | 3.06 | 10.36 | 31.58 | 125 | 5.7 | nd |
| 25 | A2 (96) | B2 (3) | B3 (1) | 35 | 23 | 49 | 3.20 | 10.66 | 31.81 | 124 | 5.2 | 68 |
| 26 | A3 (98) | B3 (2) | — | 47 | 44 | 49 | 2.29 | 9.78 | 26.02 | 93 | 10.6 | nd |
| 27 | A3 (97) | B3 (3) | — | 47 | 44 | 49 | 2.29 | 9.72 | 26.24 | 92 | 12.9 | nd |
| 28 | A3 (96) | B3 (4) | — | 47 | 44 | 49 | 2.27 | 9.70 | 26.63 | 90 | 13.9 | nd |
| 29R | A3 (80) | B3 (20) | — | 47 | 44 | 49 | 2.08 | 8.54 | 25.09 | 85 | 26.2 | nd |
| 30 | A3 (96) | B1 (4) | — | 22 | 14 | 63 | 2.09 | 10.18 | 28.97 | 89 | 7.3 | 96 |
| 31 | A3 (96) | B2 (4) | — | 36 | 26 | 68 | 2.11 | 9.66 | 27.17 | 88 | 10.5 | nd |

Cleaning Performance—Abrasive Silica Particles

Ferric Tannate (FT) Cleaning Test

Table 3 provides data for FT Cleaning Tests performed for each abrasive silica A1, A2, A3, B1, B2, B3 and B4, and for each of abrasive silica particles examples 1-31. The FT Cleaning Tests were performed in accordance with the methods described above for 100 strokes (Table 3). The change in cleaning performance of each example relative to the first “bulk” abrasive silica (A1, A2, A3) is provided as ‘% Increase Relative to First Silica’. The cleaning performance of each example relative to reference examples comprising 20 wt % of a second abrasive silica is provided as “% Cleaning Performance relative to 20 wt % loading”. These data reveal that particular silica combinations, having particular properties, enable the amount of second abrasive silica included in the abrasive silica particles to be reduced significantly, without compromising on cleaning performance.

TABLE 3

| | | | FT (100 Strokes) Cleaning @3.3% loading | | |
|---|---|---|---|---|---|
| | Wt | | | % Increase | % Cleaning |
| | % | | | Relative to | Performance relative |
| Silica | B1-B4 | PAV | FT (100) | First Silica | to 20 wt % loading |
| A1 | — | 3.8 | 44.0 | — | — |
| A2 | — | 3.4 | 39.3 | — | — |
| A3 | — | 6.5 | 70.1 | — | — |
| B1 | — | 8.5 | 86.7 | — | — |
| B2 | — | 20.9 | 90.3 | — | — |
| B3 | — | 36.7 | 94.2 | — | — |
| B4 | — | 18.5 | 83.2 | — | — |
| Ex. 1 | 2 | 4.2 | 63.3 | 43.9 | 76.1 |
| Ex. 2 | 3 | 4.4 | 69.5 | 58.0 | 83.5 |
| Ex. 3 | 4 | 4.7 | 69.5 | 58.0 | 83.5 |
| Ex. 4R | 20 | 5.9 | 83.2 | 111.7 | — |
| Ex. 5 | 1 | 4.1 | 50.8 | 29.3 | 57.3 |
| Ex. 6 | 3 | 4.2 | 70.0 | 78.1 | 79.0 |
| Ex. 7 | 5 | 4.4 | 78.3 | 99.2 | 88.4 |
| Ex. 8 | 7 | 4.4 | 78.1 | 98.7 | 88.1 |
| Ex. 9 | 10 | 4.7 | 86.4 | 119.8 | 97.5 |
| Ex. 10R | 13 | 5.3 | 88.8 | 126.0 | 100.2 |
| Ex. 11R | 16 | 5.6 | 91.7 | 133.3 | 103.5 |
| Ex. 12R | 20 | 5.5 | 88.6 | 125.4 | — |
| Ex. 13 | 2 | 7.2 | 69.8 | 58.6 | 75.3 |
| Ex. 14 | 3 | 9.9 | 71.6 | 62.7 | 77.2 |
| Ex. 15 | 4 | 13.3 | 77.9 | 77.0 | 84.0 |
| Ex. 16R | 20 | 26.5 | 92.7 | 110.7 | — |
| Ex. 17 | 2 | 5.2 | 81.3 | 84.8 | 92.6 |
| Ex. 18 | 3 | 7.9 | 82.6 | 87.7 | 94.1 |
| Ex. 19 | 4 | 9.4 | 87.6 | 99.1 | 99.8 |
| Ex. 20R | 20 | 13.3 | 87.8 | 99.5 | — |
| Ex. 21R | 4 | 4.2 | 38.5 | −12.5 | 99.2 |
| Ex. 22R | 20 | 4.2 | 38.8 | −11.8 | — |
| Ex. 23R | 4 | 3.8 | 47.4 | 7.7 | 86.5 |
| Ex. 24R | 20 | 5.7 | 54.8 | 24.5 | — |
| Ex. 25 | 4 | 5.2 | 73.4 | 86.8 | — |
| | (total) | | | | |
| Ex. 26 | 2 | 10.6 | 77.4 | 10.4 | 85.1 |
| Ex. 27 | 3 | 12.9 | 88.0 | 25.5 | 96.8 |
| Ex. 28 | 4 | 13.9 | 89.4 | 27.5 | 98.3 |
| Ex. 29R | 20 | 26.2 | 90.9 | 29.7 | — |
| Ex. 30 | 4 | 7.3 | 76.7 | 9.4 | — |
| Ex. 31 | 4 | 10.5 | 77.5 | 10.6 | — |

These data show that examples of the invention are able to deliver excellent cleaning performance, similar to that delivered by reference examples containing 20 wt % second silica, despite up to a 10-fold decrease loading of the second silica. Examples 17-19 (B2+A1) were found to provide from 90-99% of the cleaning performance of reference Example 20R comprising 20 wt % second abrasive silica, despite containing up to 10-fold less of the second silica component, and whilst advantageously delivering significantly reduced abrasion. Similarly, Examples 26-28 (B3 in A3) were found to have from 85-98% of the cleaning performance of reference Example 29R comprising 20 wt % second abrasive silica. Similar results were observed for Examples 13-15 relative to reference Example 16R, for Examples 5-9 relative to reference Example 12R, and for reference Examples 1-3 relative to reference Example 4R.

These data also demonstrate that examples of the invention provide significantly improved cleaning performance relative to the first abrasive silica alone using relatively low amounts of second silica (less than 10 wt % e.g. 2, 3, 4, 5 or 7 wt). Examples 17-19 (comprising 2, 3 and 4 wt % B2 respectively) reveal that the improvement in cleaning performance (FT₁₀₀) relative to the first abrasive silica (A1 alone) was 84-99%, which was similar to the improvement provided by reference Example 20R (20 wt % B2; 99.5%). Similarly, Examples 27 and 28 reveal that the improvement in cleaning performance relative to the first abrasive silica was 25-28%, which was similar to the improvement provided by reference Example 29R (20 wt % second silica, 29.7%). Similar results were observed for Examples 13-15 relative to reference Example 16R, for Examples 5-9 relative to reference Example 12R, and for reference Examples 1-3 relative to reference Example 4R.

These data further demonstrate that by controlling the particle size of the second silica relative to the first, excellent cleaning can be afforded whilst using comparatively low amounts of second silica. Reference Examples 23R and 24R fall outside the claimed invention because the relative particle sizes of the first and second silicas are not in accordance with the invention. These data show that for such samples, the improvement in cleaning performance at 20 wt % loading of second silica is not maintained at lower loadings of the second silica, unlike abrasive silicas of the invention. Comparing Example 23R (4 wt % B4) with Example 24R (20 wt % B4) it can be seen that the improvement in cleaning performance relative to the first silica (A1 alone) is merely 7.7% for Example 23R compared to the 24.5% improvement provided by Example 24R. Providing 4 wt % B4 therefore provides only a modest increase in cleaning performance (7.7%, Example 23R) relative to the improvement provided by 20 wt % B4 (24.5%, Example 24R). These data support that in order to achieve good cleaning performance, the relative properties of the first and second silica must be carefully controlled.

Example 25 is provided to show that good cleaning performance can also be achieved (86% relative to the first silica A2 alone) where a second silica (B2) and a third silica (B3) are provided in a total amount of 4 wt % relative to the total weight of the abrasive silica particles.

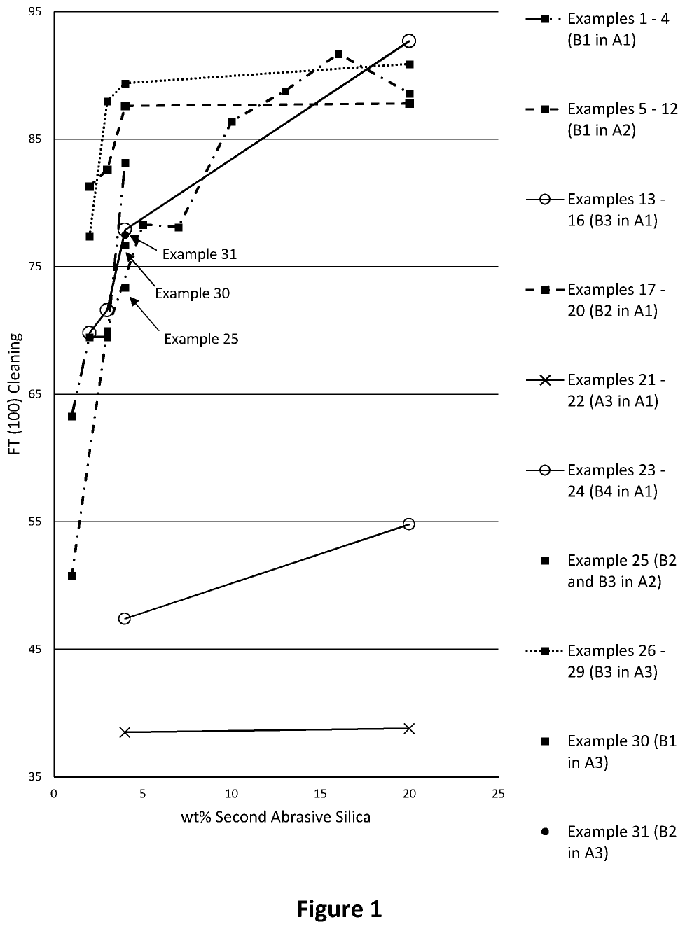

FIG. 1 illustrates the FT Cleaning Test (100) performance of Examples 1-31. Examples of abrasive silica particles having particular combinations of first “bulk” abrasive silica and second abrasive silica, provide good cleaning properties even at very low amounts of second abrasive silica (B1, B2, B3, B4), as described above.

Cleaning Performance Versus Abrasivity

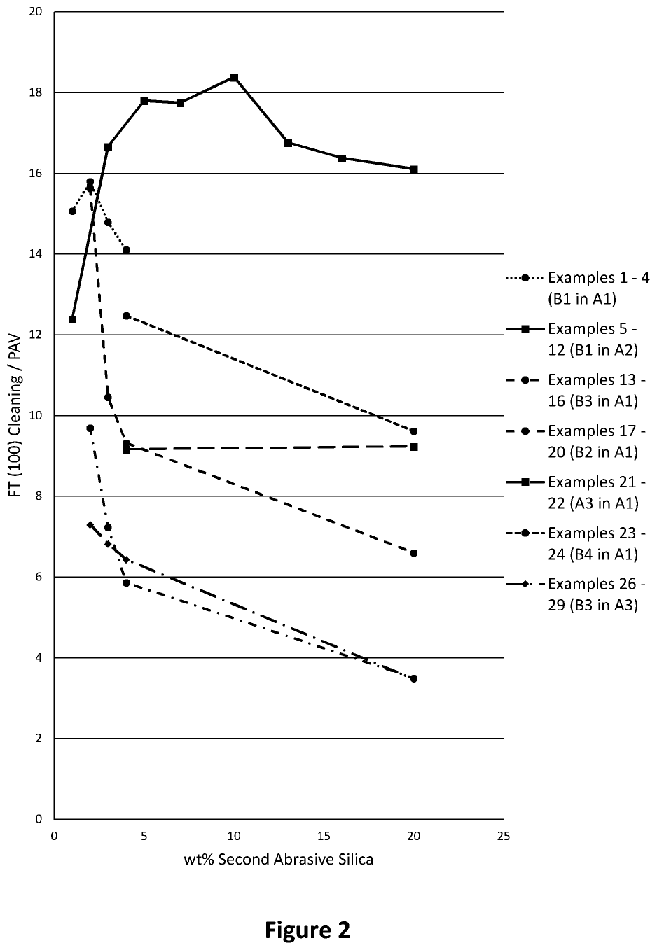

The FT₁₀₀ of Examples 1-31 relative to the PAV of each example is provided in Table 4 below (FT₁₀₀/PAV; cleaning to abrasiveness ratio). The FT₁₀₀/PAV for abrasive silicas A1-A3 and B1-B4 are also provided. The FT₁₀₀/PAV data is illustrated in FIG. 2.

As previously discussed, it is desirable to provide abrasive silica particles with good cleaning properties, but not so abrasive as to cause undesirable damage to a tooth surface. It is therefore desirable to provide abrasive silica particles, which provide excellent cleaning (high FT₁₀₀), whilst simultaneously delivering low abrasivity (e.g. low PAV). In other words, it is desirable for the FT₁₀₀/PAV ratio to be higher rather than lower.

TABLE 4
Cleaning performance (FT₁₀₀)
relative to abrasiveness (PAV) ratio

| | | FT (100 Strokes) | FT(100)/ |
|---|---|---|---|
| Silica | PAV | Cleaning | PAV |
| A1 | 3.8 | 44 | 11.6 |
| A2 | 3.4 | 39.3 | 11.6 |
| A3 | 6.5 | 70.1 | 10.8 |
| B1 | 8.5 | 86.7 | 10.2 |
| B2 | 20.9 | 90.3 | 4.3 |
| B3 | 36.7 | 94.2 | 2.6 |
| B4 | 18.5 | 83.2 | 4.5 |
| Ex. 1 | 4.2 | 63.3 | 15.1 |
| Ex. 2 | 4.4 | 69.5 | 15.8 |
| Ex. 3 | 4.7 | 69.5 | 14.8 |
| Ex. 4R | 5.9 | 83.2 | 14.1 |
| Ex. 5 | 4.1 | 50.8 | 12.4 |
| Ex. 6 | 4.2 | 70 | 16.7 |
| Ex. 7 | 4.4 | 78.3 | 17.8 |
| Ex. 8 | 4.4 | 78.1 | 17.8 |
| Ex. 9 | 4.7 | 86.4 | 18.4 |
| Ex. 10R | 5.3 | 88.8 | 16.8 |
| Ex. 11R | 5.6 | 91.7 | 16.4 |
| Ex. 12R | 5.5 | 88.6 | 16.1 |
| Ex. 13 | 7.2 | 69.8 | 9.7 |
| Ex. 14 | 9.9 | 71.6 | 7.2 |
| Ex. 15 | 13.3 | 77.9 | 5.9 |
| Ex. 16R | 26.5 | 92.7 | 3.5 |
| Ex. 17 | 5.2 | 81.3 | 15.6 |
| Ex. 18 | 7.9 | 82.6 | 10.5 |
| Ex. 19 | 9.4 | 87.6 | 9.3 |
| Ex. 20R | 13.3 | 87.8 | 6.6 |
| Ex. 21R | 4.2 | 38.5 | 9.2 |
| Ex. 22R | 4.2 | 38.8 | 9.2 |
| Ex. 23R | 3.8 | 47.4 | 12.5 |
| Ex. 24R | 5.7 | 54.8 | 9.6 |
| Ex. 25 | 5.2 | 73.4 | 14.1 |
| Ex. 26 | 10.6 | 77.4 | 7.3 |
| Ex. 27 | 12.9 | 88 | 6.8 |
| Ex. 28 | 13.9 | 89.4 | 6.4 |
| Ex. 29R | 26.2 | 90.9 | 3.5 |
| Ex. 30 | 7.3 | 76.7 | 10.5 |
| Ex. 31 | 10.5 | 77.5 | 7.4 |

With reference to Table 4 and FIG. 2, Examples 1-3 (2, 3 and 4 wt % B1 in A1 respectively) have a higher FT₁₀₀/PAV than reference Example 4R (20 wt % B1 in A1). Example 6-9 (3, 5, 7, 10 wt % B1 in A2 respectively) have a higher FT₁₀₀/PAV than reference Example 12R (20 wt % B1 in A1). Examples 13-15 (2, 3, 4 wt % B3 in A1 respectively) have a higher FT₁₀₀/PAV than reference Example 16R (20 wt % B3 in A1). Notably, the FT₁₀₀/PAV of Example 13 (2 wt % B3 in A1) is around 3-fold greater than Example 16R, and the FT₁₀₀/PAV of Examples 14 and 15 (3 and 4 wt % B3 in A1 respectively) are around 2-fold greater than Example 16R. Examples 17-19 (2, 3, 4 wt % B2 in A1) have a higher FT₁₀₀/PAV than Example 20R. Notably, the FT₁₀₀/PAV of Example 17 is around 2-fold greater than example 20R. Examples 26-28 (2, 3, 4 wt % B3 in A3) have a higher FT₁₀₀/PAV than Example 29R. Notably, the FT₁₀₀/PAV of Example 26 is around 4-fold greater than Example 29R, and the FT₁₀₀/PAV of Examples 26-28 is around 2-fold greater than Example 29R.

All of these data surprisingly show the Examples exemplified herein according to the invention generally provide more cleaning with less abrasion (as indicated by a lower PAV), as compared to examples comprising conventional amounts of second silica (i.e. around 20 wt %). In other words, the data reveal that comparatively better cleaning performance to abrasiveness ratio for these examples is generally provided where the amount of second abrasive silica in the abrasive silica particles according to the invention is less than 10%.

PCR

The cleaning performance of each of Examples 1 and 3 (comprising 2 wt % and 4 wt % of B1 in A1 respectively) relative to the first abrasive silica A1 was assessed using the PCR method outlined above. Similarly, the cleaning performance of Example 30 (comprising 4 wt % B1 in A3) relative to the first abrasive silica A3 was compared. The results are set out in Table 5 below.

TABLE 5

| Sample | mPCR60 | mPCR120 | mPCR360 | PCR800 | mPCR1200 |
|---|---|---|---|---|---|
| A1 | 53 | 54 | 56 | 66 | 63 |
| Example 1 | | | | 86 | |
| Example 3 | 114 | 106 | 88 | 83 | 84 |
| A3 | 129 | 118 | 94 | 95 | 88 |
| Example | 139 | 125 | 102 | 103 | 91 |
| 30 | | | | | |

Sample slurries comprising abrasive silica particles containing 2 and 4 wt % second abrasive silica B1 (Examples 1 and 3 respectively) had improved cleaning performance relative to the first “bulk” abrasive silica A1. It was surprisingly found that the improvement in cleaning was greatest at low brush strokes (e.g. 60, 120 and 360) where the increase in cleaning performance was over double the cleaning performance of A1 alone (see Example 3 mPCR60 for example). Similarly, Example 30 (comprising 4 wt % B1 in A3), had improved cleaning performance relative to the first “bulk” abrasive silica A3 at all brush strokes investigated. Similarly, the improvement in cleaning for Example 30 appeared to be more pronounced (i.e. there was a bigger increase) at low brush strokes (e.g. 60, 120, 360) relative to sample A3 alone, when compared to higher brush strokes (e.g. 800, 1200).

Cleaning Performance—Dentifrice Comprising Abrasive Silica Particles

PCR

The PCR cleaning performance of dentifrices P1, P2, P3 and P4 comprising abrasive silica particles were also investigated. Dentifrices P1-P4 set out in Table 6 were prepared according to the methods outlined above. The results are provided in Table 6.

TABLE 6

| | | RDA | | | | | |
|---|---|---|---|---|---|---|---|
| Dentifrice | Silica | Dentifrice | mPCR60 | mPCR120 | mPCR360 | PCR800 | mPCR1200 |
| P1 | A2 | 50 | 58 | 56 | 46 | 51 | 61 |
| P2 | Example 3 | 58 | 70 | 64 | 59 | 65 | 67 |
| P3 | A3 | 93 | 105 | 101 | 85 | 81 | 86 |
| P4 | Example 30 | 99 | 118 | 109 | 91 | 86 | 83 |

As can be seen in Table 6, dentifrices P2 and P4 were found to have improved cleaning properties (a higher mPCR value) relative to dentifrices P1 and P3, which comprises only the first “bulk” abrasive silica. The greatest improvement in cleaning performance relative to the first “bulk” abrasive silica was observed for low brush strokes (e.g. mPCR60 for P2 was 12 greater than mPCR60 of P1). These data again demonstrate that abrasive silica particles comprising particular combinations of abrasive silicas, wherein the amount of second abrasive silica less than 10 wt %, provide favourable cleaning properties at low brush strokes.

FT

The cleaning performance of each of dentifrices P1, P2, P3 and P4 was also assessed, using the FT Cleaning Test as outlined above. The results are set out in Table 7.

TABLE 7

| | FT Cleaning | |
|---|---|---|
| Dentifrice | 100 strokes | 300 strokes |
| P1 | 15.3 | 30.8 |
| P2 | 34.6 | 57.4 |
| P3 | 32.4 | 69.9 |
| P4 | 50.3 | 75.6 |

As can be seen in Table 7, P2 demonstrated over double the cleaning performance of P1 at 100 strokes; and almost double the cleaning performance of P1 at 300 strokes. This improvement was less pronounced for P4 compared to P3, but a significant improvement in cleaning was still observed, particularly at 100 brush strokes. Again, these data suggest that the improvement in cleaning performance provided by abrasive silica particles in accordance with the present invention is most pronounced for low brush strokes.

Relative Properties—Particles of Abrasive Silicas

Tables 8 and 9 compare some the properties of first abrasive silicas A1-A3 with second abrasive (and optionally third abrasive) silicas B1-B4. Combinations providing abrasive silica particles in accordance with the present invention are indicated in bold.

TABLE 8
Select relative properties of the second abrasive
silica relative to the first abrasive silica. Table 8
presents the d₁₀, d₅₀, d₉₀, and oil absorption
value of second abrasive silicas (B1, B2. B3. B4)
relative to the d₁₀, d₅₀, d₉₀, and oil absorption
value of a first abrasive silica (A1, A2, A3) as
a percentage e.g. for A1 + B1, the B1 silica
has a d50 which is 20.4% of the d50 for A1.

| Combination | d10 (%) | d50 (%) | d90% | oil abs (%) |
|---|---|---|---|---|
| A1 + B1 | 35.4 | 20.4 | 12.7 | 46.2 |
| A1 + B2 | 44.7 | 33.2 | 24.0 | 50.0 |
| A1 + B3 | 44.7 | 44.3 | 41.3 | 36.2 |
| A1 + B4 | 74.2 | 74.3 | 64.2 | 78.5 |
| A2 + B1 | 35.8 | 21.2 | 12.3 | 44.8 |
| A2 + B2 | 45.2 | 34.5 | 23.1 | 48.5 |
| A2 + B3 | 45.2 | 46.1 | 39.7 | 35.1 |
| A2 + B4 | 74.8 | 77.4 | 61.9 | 76.1 |
| A3 + B1 | 52.4 | 21.8 | 13.7 | 62.5 |
| A3 + B2 | 66.2 | 35.5 | 25.7 | 67.7 |
| A3 + B3 | 66.2 | 47.4 | 44.3 | 49.0 |
| A3 + B4 | 109.8 | 79.5 | 68.9 | 106.3 |

TABLE 9
Select relative properties of the
first abrasive silica relative to
the second abrasive silica. Table 9
presents the PAV and RDA of first
abrasive silicas (A1, A2, A3)
relative to the PAV and RDA of a
second abrasive silica (B1, B2. B3.
B4) as a percentage, e.g. for A1 + B1,
the A1 silica has a PAV which is
44.7% of the PAV for B1.

| | PAV | RDA |
|---|---|---|
| Combination | (%) | (%) |
| A1 + B1 | 44.7 | 34.4 |
| A1 + B2 | 18.2 | 30.6 |
| A1 + B3 | 10.4 | 19.5 |
| A1 + B4 | 20.5 | 34.6 |
| A2 + B1 | 40.0 | 33.8 |
| A2 + B2 | 16.3 | 30.1 |
| A2 + B3 | 9.3 | 19.1 |
| A2 + B4 | 18.4 | 34.0 |
| A3 + B1 | 76.5 | 67.5 |
| A3 + B2 | 31.1 | 60.1 |
| A3 + B3 | 17.7 | 38.2 |
| A3 + B4 | 35.1 | 68.0 |

Although the present invention has been described in detail, it should be understood that various changes, substitutions, and alterations are contemplated without departing from the principle and scope of the invention. Accordingly, the scope of the present invention defined herein and particularly the following claims should be interpreted in consideration of the appropriate equivalents. The terms “a”, “an” and “the” do not preclude the presence of multiple referents, unless the context clearly dictates otherwise. Optional or optionally means that the feature or activity may or may not be present. Either is contemplated. In embodiments, the optional feature or features may be present. Alternatively, the optional feature or features may not be present. Ranges may be expressed herein as “from” one particular value, and/or “to” another particular value, which is intended to be inclusive of the end-points of the range.

1. Abrasive silica particles suitable for use in a dentifrice composition, the particles comprising:
particles of a first abrasive silica in an amount of at least 90 wt % relative to the total weight of the abrasive silica particles; and
particles of a second abrasive silica in an amount of up to 10 wt % relative to the total weight of the abrasive silica particles;
wherein the weight median particle diameter (d50) of the particles of the second abrasive silica is less than the weight median particle diameter (d₅₀) of the particles of the first abrasive silica and from 15% to 70% of the d50 value of the particles of the first abrasive silica, wherein the Relative Dentine Abrasion (RDA) value of the second silica particles is greater than the RDA value of the first silica particles.

2. The abrasive silica particles according to claim 1, wherein the oil absorption value of the particles of second abrasive silica is less than the oil absorption value of the particles of the first abrasive silica, and is from 30% to 70% of the oil absorption value of the particles of the first abrasive silica.

3. The abrasive silica particles according to claim 1, wherein the weight median particle diameter (d50) of the second silica particles is from 15% to 65% of the d₅₀ of the first silica particles.

4. (canceled)

5. The abrasive silica particles according to claim 1, wherein the first silica particles have a Relative Dentine Abrasion (RDA) value that is from 10% to 70% of the RDA value of the second silica particles.

6. The abrasive silica particles according to claim 1, wherein the first silica particles have an RDA value of from 30 to 110.

7. The abrasive silica particles according to claim 1 wherein the second silica particles have an RDA value of from 120 to 300.

8. The abrasive silica particles according to claim 1, wherein the second silica particles are present in an amount of up to 7 wt % relative to the total weight of the abrasive silica particles.

9. (canceled)

10. The abrasive silica particles according to claim 1, wherein the d₅₀ of the first silica particles is 15 μm or less.

11. The abrasive silica particles according to claim 1, wherein the first silica particles have a d90 value, wherein 90% by weight of particles comprised in the first silica particles has a diameter less than the d90 value, of 35 μm or less.

12. (canceled)

13. The abrasive silica particles according to claim 1, wherein the second silica particles have a d90 value, wherein 90% by weight of particles comprised in the second silica particles has a diameter less than the d90 value, of 25 μm or less.

14. (canceled)

15. The abrasive silica particles according to claim 1, wherein the second silica particles have an oil absorption value of 120 g/100 g or less.

16. The abrasive silica particles according to claim 1, to wherein the first abrasive silica particles have an oil absorption value of 150 g/100 g or less.

17. The abrasive silica particles according to claim 1, wherein the first silica particles have a Plastic Abrasion Value (PAV) of from 2 to 8, and wherein the PAV of the first silica particles is less than the PAV of the second silica particles.

18. The abrasive silica particles according to claim 1, wherein the second silica particles have a Plastic Abrasion Value (PAV) of from 7 to 50, and wherein the PAV of the second silica particles is greater than the PAV of the first silica particles.

19. The abrasive silica particles according to claim 1, wherein the abrasive particles further comprise particles of a third abrasive silica, wherein the weight median particle diameter (d50) of the particles of the third silica is less than the d50 of the first silica particles and from 15% to 70% of the d50 of the first silica particles, and the combined weight of the particles of the second and third silicas does not exceed 10 wt % relative to the total weight of the abrasive silica particles.

20. (canceled)

21. (canceled)

22. (canceled)

23. The abrasive silica particles according to claim 1 wherein the abrasive silica particles have an FT100 cleaning value of from 40 to 100.

24. A composition for use in a dentifrice, the composition comprising the abrasive silica particles according to claim 1, and optionally a carrier.

25. (canceled)

26. (canceled)

27. A dentifrice comprising the abrasive silica particles according to claim 1.

28. (canceled)

29. (canceled)

30. A method of preparing abrasive silica particles for use in a dentifrice composition, the method comprising:
combining particles of a first abrasive silica and particles of a second abrasive silica to provide the abrasive silica particles,
wherein the particles of the first abrasive silica are present in an amount of at least 90 wt % relative to the total weight of the abrasive silica particles, and the particles of the second abrasive silica are present in an amount of up to 10 wt % relative to the total weight of the abrasive silica particles; and
wherein the weight median particle diameter (d50) of the second abrasive silica is less than the first abrasive silica, and is from 15% to 70% of the d₅₀ value of the first silica particles and
wherein the Relative Dentine Abrasion (RDA) value of the second silica particles is greater than the RDA value of the first silica particles.

31. A method according to claim 30, wherein the combining step comprises combining particles of the first abrasive silica and the second abrasive silica and also particles of a third abrasive silica, to provide the abrasive silica particles,
wherein the weight median particle diameter d₅₀ of the particles of the third abrasive silica is less than the first abrasive silica particles and from 15% to 70% of the weight median particle diameter d₅₀ of the first abrasive silica particles, and the combined weight of the particles of the second and third abrasive silicas does not exceed 10 wt % relative to the total weight of the abrasive silica particles.

32. (canceled)

33. (canceled)

34. (canceled)

35. A dentifrice comprising a composition according to claim 24.
